SEQUENCE LISTING (24 sequences)

SEQ ID NO: 1 - DNA, 2554 bp, Homo sapiens
acaaggcagcctcgctcgagcgcaggccaatcggctttctagctagagggtttaactcctatttaaaaagaagaacctttgaattctaacggctgagctcttggaagacttgggtccttgggtcgcaggtgggagccgacgggtgggtagaccgtgggggatatctcagtggcggacgaggacggcggggacaaggggcggctggtcggagtggcggagcgtcaagtcccctgtcggttcctccgtccctgagtgtccttggcgctgccttgtgcccgcccagcgcctttgcatccgctcctgggcaccgaggcgccctgtaggatactgcttgttacttattacagctagagggtctcactccattgcccaggccagagtgcggggatatttgataagaaacttcagtgaaggccgggcgcggtggctcatgcccgtaatcccagcattttcggaggccgaggctggagtgcaatggtgtgatctcagctcactgcaacctctgcttcctgggtttaagtgattctcctgcctcagcctcccgagtagctgggattacaggcatcatggaccgatctaaagaaaactgcatttcaggacctgttaaggctacagctccagttggaggtccaaaacgtgttctcgtgactcagcaatttccttgtcagaatccattacctgtaaatagtggccaggctcagcgggtcttgtgtccttcaaattcttcccagcgcattcctttgcaagcacaaaagcttgtctccagtcacaagccggttcagaatcagaagcagaagcaattgcaggcaaccagtgtacctcatcctgtctccaggccactgaataacacccaaaagagcaagcagcccctgccatcggcacctgaaaataatcctgaggaggaactggcatcaaaacagaaaaatgaagaatcaaaaaagaggcagtgggctttggaagactttgaaattggtcgccctctgggtaaaggaaagtttggtaatgtttatttggcaagagaaaagcaaagcaagtttattctggctcttaaagtgttatttaaagctcagctggagaaagccggagtggagcatcagctcagaagagaagtagaaatacagtcccaccttcggcatcctaatattcttagactgtatggttatttccatgatgctaccagagtctacctaattctggaatatgcaccacttggaacagtttatagagaacttcagaaactttcaaagtttgatgagcagagaactgctacttatataacagaattggcaaatgccctgtcttactgtcattcgaagagagttattcatagagacattaagccagagaacttacttcttggatcagctggagagcttaaaattgcagattttgggtggtcagtacatgctccatcttccaggaggaccactctctgtggcaccctggactacctgccccctgaaatgattgaaggtcggatgcatgatgagaaggtggatctctggagccttggagttctttgctatgaatttttagttgggaagcctccttttgaggcaaacacataccaagagacctacaaaagaatatcacgggttgaattcacattccctgactttgtaacagagggagccagggacctcatttcaagactgttgaagcataatcccagccagaggccaatgctcagagaagtacttgaacacccctggatcacagcaaattcatcaaaaccatcaaattgccaaaacaaagaatcagctagcaaacagtcttaggaatcgtgcagggggagaaatccttgagccagggctgccatataacctgacaggaacatgctactgaagtttattttaccattgactgctgccctcaatctagaacgctacacaagaaatatttgttttactcagcaggtgtgccttaacctccctattcagaaagctccacatcaataaacatgacactctgaagtgaaagtagccacgagaattgtgctacttatactggttcataatctggaggcaaggttcgactgcagccgccccgtcagcctgtgctaggcatggtgtcttcacaggaggcaaatccagagcctggctgtggggaaagtgaccactctgccctgaccccgatcagttaaggagctgtgcaataaccttcctagtacctgagtgagtgtgtaacttattgggttggcgaagcctggtaaagctgttggaatgagtatgtgattctttttaagtatgaaaataaagatatatgtacagacttgtattttttctctggtggcattcctttaggaatgctgtgtgtctgtccggcaccccggtaggcctgattgggtttctagtcctccttaaccacttatctcccatatgagagtgtgaaaaataggaacacgtgctctacctccatttagggatttgcttgggatacagaagaggccatgtgtctcagagctgttaagggcttatttttttaaaacattggagtcatagcatgtgtgtaaactttaaatatgcaaataaataagtatctatgtctaaaaaaa

SEQ ID NO: 2 - DNA, 8680 bp, Homo sapiens
acggggcctgaagcggcggtaccggtgctggcggcggcagctgaggccttggccgaagccgcgcgaacctcagggcaagatgcttggaaccggacctgccgccgccaccaccgctgccaccacatctagcaatgtgagcgtcctgcagcagtttgccagtggcctaaagagccggaatgaggaaaccagggccaaagccgccaaggagctccagcactatgtcaccatggaactccgagagatgagtcaagaggagtctactcgcttctatgaccaactgaaccatcacatttttgaattggtttccagctcagatgccaatgagaggaaaggtggcatcttggccatagctagcctcataggagtggaaggtgggaatgccacccgaattggcagatttgccaactatcttcggaacctcctcccctccaatgacccagttgtcatggaaatggcatccaaggccattggccgtcttgccatggcaggggacacttttaccgctgagtacgtggaatttgaggtgaagcgagccctggaatggctgggtgctgaccgcaatgagggccggagacatgcagctgtcctggttctccgtgagctggccatcagcgtccctaccttcttcttccagcaagtgcaacccttctttgacaacatttttgtggccgtgtgggaccccaaacaggccatccgtgagggagctgtagccgcccttcgtgcctgtctgattctcacaacccagcgtgagccgaaggagatgcagaagcctcagtggtacaggcacacatttgaagaagcagagaagggatttgatgagaccttggccaaagagaagggcatgaatcgggatgatcggatccatggagccttgttgatccttaacgagctggtccgaatcagcagcatggagggagagcgtctgagagaagaaatggaagaaatcacacagcagcagctggtacacgacaagtactgcaaagatctcatgggcttcggaacaaaacctcgtcacattacccccttcaccagtttccaggctgtacagccccagcagtcaaatgccttggtggggctgctggggtacagctctcaccaaggcctcatgggatttgggacctcccccagtccagctaagtccaccctggtggagagccggtgttgcagagacttgatggaggagaaatttgatcaggtgtgccagtgggtgctgaaatgcaggaatagcaagaactcgctgatccaaatgacaatccttaatttgttgccccgcttggctgcattccgaccttctgccttcacagatacccagtatctccaagataccatgaaccatgtcctaagctgtgtcaagaaggagaaggaacgtacagcggccttccaagccctggggctactttctgtggctgtgaggtctgagtttaaggtctatttgcctcgcgtgctggacatcatccgagcggccctgcccccaaaggacttcgcccataagaggcagaaggcaatgcaggtggatgccacagtcttcacttgcatcagcatgctggctcgagcaatggggccaggcatccagcaggatatcaaggagctgctggagcccatgctggcagtgggactaagccctgccctcactgcagtgctctacgacctgagccgtcagattccacagctaaagaaggacattcaagatgggctactgaaaatgctgtccctggtccttatgcacaaaccccttcgccacccaggcatgcccaagggcctggcccatcagctggcctctcctggcctcacgaccctccctgaggccagcgatgtgggcagcatcactcttgccctccgaacgcttggcagctttgaatttgaaggccactctctgacccaatttgttcgccactgtgcggatcatttcctgaacagtgagcacaaggagatccgcatggaggctgcccgcacctgctcccgcctgctcacaccctccatccacctcatcagtggccatgctcatgtggttagccagaccgcagtgcaagtggtggcagatgtgcttagcaaactgctcgtagttgggataacagatcctgaccctgacattcgctactgtgtcttggcgtccctggacgagcgctttgatgcacacctggcccaggcggagaacttgcaggccttgtttgtggctctgaatgaccaggtgtttgagatccgggagctggccatctgcactgtgggccgactcagtagcatgaaccctgcctttgtcatgcctttcctgcgcaagatgctcatccagattttgacagagttggagcacagtgggattggaagaatcaaagagcagagtgcccgcatgctggggcacctggtctccaatgccccccgactcatccgcccctacatggagcctattctgaaggcattaattttgaaactgaaagatccagaccctgatccaaacccaggtgtgatcaataatgtcctggcaacaataggagaattggcacaggttagtggcctggaaatgaggaaatgggttgatgaactttttattatcatcatggacatgctccaggattcctctttgttggccaaaaggcaggtggctctgtggaccctgggacagttggtggccagcactggctatgtagtagagccctacaggaagtaccctactttgcttgaggtgctactgaattttctgaagactgagcagaaccagggtacacgcagagaggccatccgtgtgttagggcttttaggggctttggatccttacaagcacaaagtgaacattggcatgatagaccagtcccgggatgcctctgctgtcagcctgtcagaatccaagtcaagtcaggattcctctgactatagcactagtgaaatgctggtcaacatgggaaacttgcctctggatgagttctacccagctgtgtccatggtggccctgatgcggatcttccgagaccagtcactctctcatcatcacaccatggttgtccaggccatcaccttcatcttcaagtccctgggactcaaatgtgtgcagttcctgccccaggtcatgcccacgttccttaacgtcattcgagtctgtgatggggccatccgggaatttttgttccagcagctgggaatgttggtgtcctttgtgaagagccacatcagaccttatatggatgaaatagtcaccctcatgagagaattctgggtcatgaacacctcaattcagagcacgatcattcttctcattgagcaaattgtggtagctcttgggggtgaatttaagctctacctgccccagctgatcccacacatgctgcgtgtcttcatgcatgacaacagcccaggccgcattgtctctatcaagttactggctgcaatccagctgtttggcgccaacctggatgactacctgcatttactgctgcctcctattgttaagttgtttgatgcccctgaagctccactgccatctcgaaaggcagcgctagagactgtggaccgcctgacggagtccctggatttcactgactatgcctcccggatcattcaccctattgttcgaacactggaccagagcccagaactgcgctccacagccatggacacgctgtcttcacttgtttttcagctggggaagaagtaccaaattttcattccaatggtgaataaagttctggtgcgacaccgaatcaatcatcagcgctatgatgtgctcatctgcagaattgtcaagggatacacacttgctgatgaagaggaggatcctttgatttaccagcatcggatgcttaggagtggccaaggggatgcattggctagtggaccagtggaaacaggacccatgaagaaactgcacgtcagcaccatcaacctccaaaaggcctggggcgctgccaggagggtctccaaagatgactggctggaatggctgagacggctgagcctggagctgctgaaggactcatcatcgccctccctgcgctcctgctgggccctggcacaggcctacaacccgatggccagggatctcttcaatgctgcatttgtgtcctgctggtctgaactgaatgaagatcaacaggatgagctcatcagaagcatcgagttggccctcacctcacaagacatcgctgaagtcacacagaccctcttaaacttggctgaattcatggaacacagtgacaagggccccctgccactgagagatgacaatggcattgttctgctgggtgagagagctgccaagtgccgagcatatgccaaagcactacactacaaagaactggagttccagaaaggccccacccctgccattctagaatctctcatcagcattaataataagctacagcagccggaggcagcggccggagtgttagaatatgccatgaaacactttggagagctggagatccaggctacctggtatgagaaactgcacgagtgggaggatgcccttgtggcctatgacaagaaaatggacaccaacaaggacgacccagagctgatgctgggccgcatgcgctgcctcgaggccttgggggaatggggtcaactccaccagcagtgctgtgaaaagtggaccctggttaatgatgagacccaagccaagatggcccggatggctgctgcagctgcatggggtttaggtcagtgggacagcatggaagaatacacctgtatgatccctcgggacacccatgatggggcattttatagagctgtgctggcactgcatcaggacctcttctccttggcacaacagtgcattgacaaggccagggacctgctggatgctgaattaactgcgatggcaggagagagttacagtcgggcatatggggccatggtttcttgccacatgctgtccgagctggaggaggttatccagtacaaacttgtccccgagcgacgagagatcatccgccagatctggtgggagagactgcagggctgccagcgtatcgtagaggactggcagaaaatccttatggtgcggtcccttgtggtcagccctcatgaagacatgagaacctggctcaagtatgcaagcctgtgcggcaagagtggcaggctggctcttgctcataaaactttagtgttgctcctgggagttgatccgtctcggcaacttgaccatcctctgccaacagttcaccctcaggtgacctatgcctacatgaaaaacatgtggaagagtgcccgcaagatcgatgccttccagcacatgcagcattttgtccagaccatgcagcaacaggcccagcatgccatcgctactgaggaccagcagcataagcaggaactgcacaagctcatggcccgatgcttcctgaaacttggagagtggcagctgaatctacagggcatcaatgagagcacaatccccaaagtgctgcagtactacagcgccgccacagagcacgaccgcagctggtacaaggcctggcatgcgtgggcagtgatgaacttcgaagctgtgctacactacaaacatcagaaccaagcccgcgatgagaagaagaaactgcgtcatgccagcggggccaacatcaccaacgccaccactgccgccaccacggccgccactgccaccaccactgccagcaccgagggcagcaacagtgagagcgaggccgagagcaccgagaacagccccaccccatcgccgctgcagaagaaggtcactgaggatctgtccaaaaccctcctgatgtacacggtgcctgccgtccagggcttcttccgttccatctccttgtcacgaggcaacaacctccaggatacactcagagttctcaccttatggtttgattatggtcactggccagatgtcaatgaggccttagtggagggggtgaaagccatccagattgatacctggctacaggttatacctcagctcattgcaagaattgatacgcccagacccttggtgggacgtctcattcaccagcttctcacagacattggtcggtaccacccccaggccctcatctacccactgacagtggcttctaagtctaccacgacagcccggcacaatgcagccaacaagattctgaagaacatgtgtgagcacagcaacaccctggtccagcaggccatgatggtgagcgaggagctgatccgagtggccatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatctcaaagcagctgcctcagctcacatccttagagctgcaatatgtttccccaaaacttctgatgtgccgggaccttgaattggctgtgccaggaacatatgaccccaaccagccaatcattcgcattcagtccatagcaccgtctttgcaagtcatcacatccaagcagaggccccggaaattgacacttatgggcagcaacggacatgagtttgttttccttctaaaaggccatgaagatctgcgccaggatgagcgtgtgatgcagctcttcggcctggttaacacccttctggccaatgacccaacatctcttcggaaaaacctcagcatccagagatacgctgtcatccctttatcgaccaactcgggcctcattggctgggttccccactgtgacacactgcacgccctcatccgggactacagggagaagaagaagatccttctcaacatcgagcatcgcatcatgttgcggatggctccggactatgaccacttgactctgatgcagaaggtggaggtgtttgagcatgccgtcaataatacagctggggacgacctggccaagctgctgtggctgaaaagccccagctccgaggtgtggtttgaccgaagaaccaattatacccgttctttagcggtcatgtcaatggttgggtatattttaggcctgggagatagacacccatccaacctgatgctggaccgtctgagtgggaagatcctgcacattgactttggggactgctttgaggttgctatgacccgagagaagtttccagagaagattccatttagactaacaagaatgttgaccaatgctatggaggttacaggcctggatggcaactacagaatcacatgccacacagtgatggaggtgctgcgagagcacaaggacagtgtcatggccgtgctggaagcctttgtctatgaccccttgctgaactggaggctgatggacacaaataccaaaggcaacaagcgatcccgaacgaggacggattcctactctgctggccagtcagtcgaaattttggacggtgtggaacttggagagccagcccataagaaaacggggaccacagtgccagaatctattcattctttcattggagacggtttggtgaaaccagaggccctaaataagaaagctatccagattattaacagggttcgagataagctcactggtcgggacttctctcatgatgacactttggatgttccaacgcaagttgagctgctcatcaaacaagcgacatcccatgaaaacctctgccagtgctatattggctggtgccctttctggtaactggaggcccagatgtgcccatcacgttttttctgaggcttttgtactttagtaaatgcttccactaaactgaaaccatggtgagaaagtttgactttgttaaatattttgaaatgtaaatgaaaagaactactgtatattaaaagttggtttgaaccaactttctagctgctgttgaagaatatattgtcagaaacacaaggcttgatttggttcccaggacagtgaaacaatagtaataccacgtaaatcaagccattcattttggggaacagaagatccataactttagaaatacgggttttgacttaactcacaagagaactcatcataagtacttgctgatggaagaatgacctagttgctcctctcaacatgggtacagcaaactcagcacagccaagaagcctcaggtcgtggagaacatggattaggatcctagactgtaaagacacagaagatgctgacctcacccctgccacctatcccaagacctcactggtctgtggacagcagcagaaatgtttgcaagataggccaaaatgagtacaaaaggtctgtcttccatcagacccagtgatgctgcgactcacacgcttcaattcaagacctgaccgctagtagggaggtttattcagatcgctggcagcctcggctgagcagatgcacagaggggatcactgtgcagtgggaccaccctcactggccttctgcagcagggttctgggatgttttcagtggtcaaaatactctgtttagagcaagggctcagaaaacagaaatactgtcatggaggtgctgaacacagggaaggtctggtacatattggaaattatgagcagaacaaatactcaactaaatgcacaaagtataaagtgtagccatgtctagacaccatgttgtatcagaataatttttgtgccaataaatgacatcagaattttaaacata

SEQ ID NO: 3 - DNA, 5916 bp, Homo sapiens
gcccctccctccgcccgcccgccggcccgcccgtcagtctggcaggcaggcaggcaatcggtccgagtggctgtcggctcttcagctctcccgctcggcgtcttccttcctcctcccggtcagcgtcggcggctgcaccggcggcggcgcagtccctgcgggaggggcgacaagagctgagcggcggccgccgagcgtcgagctcagcgcggcggaggcggcggcggcccggcagccaacatggcggcggcggcggcggcgggcgcgggcccggagatggtccgcgggcaggtgttcgacgtggggccgcgctacaccaacctctcgtacatcggcgagggcgcctacggcatggtgtgctctgcttatgataatgtcaacaaagttcgagtagctatcaagaaaatcagcccctttgagcaccagacctactgccagagaaccctgagggagataaaaatcttactgcgcttcagacatgagaacatcattggaatcaatgacattattcgagcaccaaccatcgagcaaatgaaagatgtatatatagtacaggacctcatggaaacagatctttacaagctcttgaagacacaacacctcagcaatgaccatatctgctattttctctaccagatcctcagagggttaaaatatatccattcagctaacgttctgcaccgtgacctcaagccttccaacctgctgctcaacaccacctgtgatctcaagatctgtgactttggcctggcccgtgttgcagatccagaccatgatcacacagggttcctgacagaatatgtggccacacgttggtacagggctccagaaattatgttgaattccaagggctacaccaagtccattgatatttggtctgtaggctgcattctggcagaaatgctttctaacaggcccatctttccagggaagcattatcttgaccagctgaaccacattttgggtattcttggatccccatcacaagaagacctgaattgtataataaatttaaaagctaggaactatttgctttctcttccacacaaaaataaggtgccatggaacaggctgttcccaaatgctgactccaaagctctggacttattggacaaaatgttgacattcaacccacacaagaggattgaagtagaacaggctctggcccacccatatctggagcagtattacgacccgagtgacgagcccatcgccgaagcaccattcaagttcgacatggaattggatgacttgcctaaggaaaagctcaaagaactaatttttgaagagactgctagattccagccaggatacagatcttaaatttgtcaggacaagggctcagaggactggacgtgctcagacatcggtgttcttcttcccagttcttgacccctggtcctgtctccagcccgtcttggcttatccactttgactcctttgagccgtttggaggggcggtttctggtagttgtggcttttatgctttcaaagaatttcttcagtccagagaattcctcctggcagccctgtgtgtgtcacccattggtgacctgcggcagtatgtacttcagtgcacctactgcttactgttgctttagtcactaattgctttctggtttgaaagatgcagtggttcctccctctcctgaatccttttctacatgatgccctgctgaccatgcagccgcaccagagagagattcttccccaattggctctagtcactggcatctcactttatgatagggaaggctactacctagggcactttaagtcagtgacagccccttatttgcacttcaccttttgaccataactgtttccccagagcaggagcttgtggaaataccttggctgatgttgcagcctgcagcaagtgcttccgtctccggaatccttggggagcacttgtccacgtcttttctcatatcatggtagtcactaacatatataaggtatgtgctattggcccagcttttagaaaatgcagtcatttttctaaataaaaaggaagtactgcacccagcagtgtcactctgtagttactgtggtcacttgtaccatatagaggtgtaacacttgtcaagaagcgttatgtgcagtacttaatgtttgtaagacttacaaaaaaagatttaaagtggcagcttcactcgacatttggtgagagaagtacaaaggttgcagtgctgagctgtgggcggtttctggggatgtcccagggtggaactccacatgctggtgcatatacgcccttgagctacttcaaatgtgggtgtttcagtaaccacgttccatgcctgaggatttagcagagaggaacactgcgtctttaaatgagaaagtatacaattctttttccttctacagcatgtcagcatctcaagttcatttttcaacctacagtataacaatttgtaataaagcctccaggagctcatgacgtgaagcactgttctgtcctcaagtactcaaatatttctgatactgctgagtcagactgtcagaaaaagctagcactaactcgtgtttggagctctatccatattttactgatctctttaagtatttgttcctgccactgtgtactgtggagttgactcggtgttctgtcccagtgcggtgcctcctcttgacttccccactgctctctgtggtgagaaatttgccttgttcaataattactgtaccctcgcatgactgttacagctttctgtgcagagatgactgtccaagtgccacatgcctacgattgaaatgaaaactctattgttacctctgagttgtgttccacggaaaatgctatccagcagatcatttaggaaaaataattctatttttagcttttcatttctcagctgtccttttttcttgtttgatttttgacagcaatggagaatgggttatataaagactgcctgctaatatgaacagaaatgcatttgtaattcatgaaaataaatgtacatcttctatcttcacattcatgttaagattcagtgttgctttcctctggatcagcgtgtctgaatggacagtcaggttcaggttgtgctgaacacagaaatgctcacaggcctcactttgccgcccaggcactggcccagcacttggatttacataagatgagttagaaaggtacttctgtagggtcctttttacctctgctcggcagagaatcgatgctgtcatgttcctttattcacaatcttaggtctcaaatattctgtcaaaccctaacaaagaagccccgacatctcaggttggattccctggttctctctaaagagggcctgcccttgtgccccagaggtgctgctgggcacagccaagagttgggaagggccgccccacagtacgcagtcctcaccacccagcccagggtgctcacgctcaccactcctgtggctgaggaaggatagctggctcatcctcggaaaacagacccacatctctattcttgccctgaaatacgcgcttttcacttgcgtgctcagagctgccgtctgaaggtccacacagcattgacgggacacagaaatgtgactgttaccggataacactgattagtcagttttcatttataaaaaagcattgacagttttattactcttgtttctttttaaatggaaagttactattataaggttaatttggagtcctcttctaaatagaaaaccatatccttggctactaacatctggagactgtgagctccttcccattccccttcctggtactgtggagtcagattggcatgaaaccactaacttcattctagaatcattgtagccataagttgtgtgctttttattaatcatgccaaacataatgtaactgggcagagaatggtcctaaccaaggtacctatgaaaagcgctagctatcatgtgtagtagatgcatcattttggctcttcttacatttgtaaaaatgtacagattaggtcatcttaattcatattagtgacacggaacagcacctccactatttgtatgttcaaataagctttcagactaatagcttttttggtgtctaaaatgtaagcaaaaaattcctgctgaaacattccagtcctttcatttagtataaaagaaatactgaacaagccagtgggatggaattgaaagaactaatcatgaggactctgtcctgacacaggtcctcaaagctagcagagatacgcagacattgtggcatctgggtagaagaatactgtattgtgtgtgcagtgcacagtgtgtggtgtgtgcacactcattccttctgctcttgggcacaggcagtgggtgtagaggtaaccagtagctttgagaagctacatgtagctcaccagtggttttctctaaggaatcacaaaagtaaactacccaaccacatgccacgtaatatttcagccattcagaggaaactgttttctctttatttgcttatatgttaatatggtttttaaattggtaacttttatatagtatggtaacagtatgttaatacacacatacatacgcacacatgctttgggtccttccataatacttttatatttgtaaatcaatgttttggagcaatcccaagtttaagggaaatatttttgtaaatgtaatggttttgaaaatctgagcaatccttttgcttatacatttttaaagcatttgtgctttaaaattgttatgctggtgtttgaaacatgatactcctgtggtgcagatgagaagctataacagtgaatatgtggtttctcttacgtcatccaccttgacatgatgggtcagaaacaaatggaaatccagagcaagtcctccagggttgcaccaggtttacctaaagcttgttgccttttcttgtgctgtttatgcgtgtagagcactcaagaaagttctgaaactgctttgtatctgctttgtactgttggtgccttcttggtattgtaccccaaaattctgcatagattatttagtataatggtaagttaaaaaatgttaaaggaagattttattaagaatctgaatgtttattcattatattgttacaatttaacattaacatttatttgtggtatttgtgatttggttaatctgtataaaaattgtaagtagaaaggtttatatttcatcttaattcttttgatgttgtaaacgtactttttaaaagatggattatttgaatgtttatggcacctgacttgtaaaaaaaaaaaactacaaaaaaatccttagaatcattaaattgtgtccctgtattaccaaaataacacagcaccgtgcatgtatagtttaattgcagtttcatctgtgaaaacgtgaaattgtctagtccttcgttatgttccccagatgtcttccagatttgctctgcatgtggtaacttgtgttagggctgtgagctgttcctcgagttgaatggggatgtcagtgctcctagggttctccaggtggttcttcagaccttcacctgtgggggggggggtaggcggtgcccacgcccatctcctcatcctcctgaacttctgcaaccccactgctgggcagacatcctgggcaaccccttttttcagagcaagaagtcataaagataggatttcttggacatttggttcttatcaatattgggcattatgtaatgacttatttacaaaacaaagatactggaaaatgttttggatgtggtgttatggaaagagcacaggccttggacccatccagctgggttcagaactaccccctgcttataactgcggctggctgtgggccagtcattctgcgtctctgctttcttcctctgcttcagactgtcagctgtaaagtggaagcaatattacttgccttgtatatggtaaagattataaaaatacatttcaactgttcagcatagtacttcaaagcaagtactcagtaaatagcaagtctttttaaa

SEQ ID NO: 4 - DNA, 2603 bp, Homo sapiens
aggcgaggcttccccttccccgcccctcccccggcctccagtccctcccagggccgcttcgcagagcggctaggagcacggcggcggcggcactttccccggcaggagctggagctgggctctggtgcgcgcgcggctgtgccgcccgagccggagggactggttggttgagagagagagaggaagggaatcccgggctgccgaaccgcacgttcagcccgctccgctcctgcagggcagcctttcggctctctgcgcgcgaagccgagtcccgggcgggtggggcgggggtccactgagaccgctaccggcccctcggcgctgacgggaccgcgcggggcgcacccgctgaaggcagccccggggcccgcggcccggacttggtcctgcgcagcgggcgcggggcagcgcagcgggaggaagcgagaggtgctgccctccccccggagttggaagcgcgttacccgggtccaaaatgcccaagaagaagccgacgcccatccagctgaacccggcccccgacggctctgcagttaacgggaccagctctgcggagaccaacttggaggccttgcagaagaagctggaggagctagagcttgatgagcagcagcgaaagcgccttgaggcctttcttacccagaagcagaaggtgggagaactgaaggatgacgactttgagaagatcagtgagctgggggctggcaatggcggtgtggtgttcaaggtctcccacaagccttctggcctggtcatggccagaaagctaattcatctggagatcaaacccgcaatccggaaccagatcataagggagctgcaggttctgcatgagtgcaactctccgtacatcgtgggcttctatggtgcgttctacagcgatggcgagatcagtatctgcatggagcacatggatggaggttctctggatcaagtcctgaagaaagctggaagaattcctgaacaaattttaggaaaagttagcattgctgtaataaaaggcctgacatatctgagggagaagcacaagatcatgcacagagatgtcaagccctccaacatcctagtcaactcccgtggggagatcaagctctgtgactttggggtcagcgggcagctcatcgactccatggccaactccttcgtgggcacaaggtcctacatgtcgccagaaagactccaggggactcattactctgtgcagtcagacatctggagcatgggactgtctctggtagagatggcggttgggaggtatcccatccctcctccagatgccaaggagctggagctgatgtttgggtgccaggtggaaggagatgcggctgagaccccacccaggccaaggacccccgggaggccccttagctcatacggaatggacagccgacctcccatggcaatttttgagttgttggattacatagtcaacgagcctcctccaaaactgcccagtggagtgttcagtctggaatttcaagattttgtgaataaatgcttaataaaaaaccccgcagagagagcagatttgaagcaactcatggttcatgcttttatcaagagatctgatgctgaggaagtggattttgcaggttggctctgctccaccatcggccttaaccagcccagcacaccaacccatgctgctggcgtctaagtgtttgggaagcaacaaagagcgagtcccctgcccggtggtttgccatgtcgcttttgggcctccttcccatgcctgtctctgttcagatgtgcatttcacctgtgacaaaggatgaagaacacagcatgtgccaagattctactcttgtcatttttaatattactgtctttattcttattactattattgttcccctaagtggattggctttgtgcttggggctatttgtgtgtatgctgatgatcaaaacctgtgccaggctgaattacagtgaaattttggtgaatgtgggtagtcattcttacaattgcactgctgttcctgctccatgactggctgtctgcctgtattttcgggattctttgacatttggtggtactttattcttgctgggcatactttctctctaggagggagccttgtgagatccttcacaggcagtgcatgtgaagcatgctttgctgctatgaaaatgagcatcagagagtgtacatcatgttattttattattattatttgcttttcatgtagaactcagcagttgacatccaaatctagccagagcccttcactgccatgatagctggggcttcaccagtctgtctactgtggtgatctgtagacttctggttgtatttctatatttattttcagtatactgtgtgggatacttagtggtatgtctctttaagttttgattaatgtttcttaaatggaattattttgaatgtcacaaattgatcaagatattaaaatgtcggatttatctttccccatatccaagtaccaatgctgttgtaaacaacgtgtatagtgcctaaaattgtatgaaaatccttttaaccattttaacctagatgtttaacaaatctaatctcttattctaataaatatactatgaaataaaaaaaaaaggatgaaagctaaaaaaaaaaaaaaaaaa

SEQ ID NO: 5 - DNA, 829 bp, Homo sapiens
cctcttttccgtggcgcctcggaggcgttcagctgcttcaagatgaagctgaacatctccttcccagccactggctgccagaaactcattgaagtggacgatgaacgcaaacttcgtactttctatgagaagcgtatggccacagaagttgctgctgacgctctgggtgaagaatggaagggttatgtggtccgaatcagtggtgggaacgacaaacaaggtttccccatgaagcagggtgtcttgacccatggccgtgtccgcctgctactgagtaaggggcattcctgttacagaccaaggagaactggagaaagaaagagaaaatcagttcgtggttgcattgtggatgcaaatctgagcgttctcaacttggttattgtaaaaaaaggagagaaggatattcctggactgactgatactacagtgcctcgccgcctgggccccaaaagagctagcagaatccgcaaacttttcaatctctctaaagaagatgatgtccgccagtatgttgtaagaaagcccttaaataaagaaggtaagaaacctaggaccaaagcacccaagattcagcgtcttgttactccacgtgtcctgcagcacaaacggcggcgtattgctctgaagaagcagcgtaccaagaaaaataaagaagaggctgcagaatatgctaaacttttggccaagagaatgaaggaggctaaggagaagcgccaggaacaaattgcgaagagacgcagactttcctctctgcgagcttctacttctaagtctgaatccagtcagaaataagattttttgagtaacaaataaataagatcagactctg

SEQ ID NO: 6 - DNA, 3008 bp, Homo sapiens
taattatgggtctgtaaccaccctggactgggtgctcctcactgacggacttgtctgaacctctctttgtctccagcgcccagcactgggcctggcaaaacctgagacgcccggtacatgttggccaaatgaatgaaccagattcagaccggcaggggcgctgtggtttaggaggggcctggggtttctcccaggaggtttttgggcttgcgctggagggctctggactcccgtttgcgccagtggcctgcatcctggtcctgtcttcctcatgtttgaatttctttgctttcctagtctggggagcagggaggagccctgtgccctgtcccaggatccatgggtaggaacaccatggacagggagagcaaacggggccatctgtcaccaggggcttagggaaggccgagccagcctgggtcaaagaagtcaaaggggctgcctggaggaggcagcctgtcagctggtgcatcagaggctgtggccaggccagctgggctcggggagcgccagcctgagaggagcgcgtgagcgtcgcgggagcctcgggcaccatgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggagcgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcggcacggcctgaggcggcggtggactgcgctggacgatagcttggagggatggagaggcggcctcgtgccatgatctgtatttaatggtttttatttctcgggtgcatttgagagaagccacgctgtcctctcgagcccagatggaaagacgtttttgtgctgtgggcagcaccctcccccgcagcggggtagggaagaaaactatcctgcgggttttaatttatttcatccagtttgttctccgggtgtggcctcagccctcagaacaatccgattcacgtagggaaatgttaaggacttctgcagctatgcgcaatgtggcattggggggccgggcaggtcctgcccatgtgtcccctcactctgtcagccagccgccctgggctgtctgtcaccagctatctgtcatctctctggggccctgggcctcagttcaacctggtggcaccagatgcaacctcactatggtatgctggccagcaccctctcctgggggtggcaggcacacagcagccccccagcactaaggccgtgtctctgaggacgtcatcggaggctgggcccctgggatgggaccagggatgggggatgggccagggtttacccagtgggacagaggagcaaggtttaaatttgttattgtgtattatgttgttcaaatgcattttgggggtttttaatctttgtgacaggaaagccctcccccttccccttctgtgtcacagttcttggtgactgtcccaccgggagcctccccctcagatgatctctccacggtagcacttgaccttttcgacgcttaacctttccgctgtcgccccaggccctccctgactccctgtgggggtggccatccctgggcccctccacgcctcctggccagacgctgccgctgccgctgcaccacggcgtttttttacaacattcaactttagtatttttactattataatataatatggaaccttccctccaaattcttcaataaaagttgcttttcaaaaaaaaaaaaaaaaaaaaaa

SEQ ID NO: 7 - DNA, 4343 bp, Homo sapiens
tggtcatcgcacggcggcagctcctcacctggatttagaagagctggcgtccccgcccgcccaagcctttaaactctcgtctgccagaacccgccaactctccaggcttagggccagtttccgcgattctaagagtaattgcgtgggcacctgtgctggggccaggcgcaaagaagggagttggtctgcgcgaagatcgtcaacctgctaacagaccgcacatgcactttgcaccgaccatctacgtctcagtctggaggttgcgcactttggctgctgacgcgctggtggtgcctattaatcatttaccagtccagagccgcgccagttaatggctgtgccgtgcggtgctcccacatcctggcctctcctctccacggtcgcctgtgcccgggcaccccggagctgcaaactgcagagcccaggcaaccgctgggctgtgcgccccgccggcgccggtaggagccgcgctccccgcagcggttgcgctctacccggaggcgctgggcggctgtgggctgcaggcaagcggtcgggtggggagggagggcgcaggcggcgggtgcgcgaggagaaagccccagccctggcagccccactggcccccctcagctgggatgttccccaatggcaccgcctcctctccttcctcctctcctagccccagcccgggcagctgcggcgaaggcggcggcagcaggggccccggggccggcgctgcggacggcatggaggagccagggcgaaatgcgtcccagaacgggaccttgagcgagggccagggcagcgccatcctgatctctttcatctactccgtggtgtgcctggtggggctgtgtgggaactctatggtcatctacgtgatcctgcgctatgccaagatgaagacggccaccaacatctacatcctaaatctggccattgctgatgagctgctcatgctcagcgtgcccttcctagtcacctccacgttgttgcgccactggcccttcggtgcgctgctctgccgcctcgtgctcagcgtggacgcggtcaacatgttcaccagcatctactgtctgactgtgctcagcgtggaccgctacgtggccgtggtgcatcccatcaaggcggcccgctaccgccggcccaccgtggccaaggtagtaaacctgggcgtgtgggtgctatcgctgctcgtcatcctgcccatcgtggtcttctctcgcaccgcggccaacagcgacggcacggtggcttgcaacatgctcatgccagagcccgctcaacgctggctggtgggcttcgtgttgtacacatttctcatgggcttcctgctgcccgtgggggctatctgcctgtgctacgtgctcatcattgctaagatgcgcatggtggccctcaaggccggctggcagcagcgcaagcgctcggagcgcaagatcaccttaatggtgatgatggtggtgatggtgtttgtcatctgctggatgcctttctacgtggtgcagctggtcaacgtgtttgctgagcaggacgacgccacggtgagtcagctgtcggtcatcctcggctatgccaacagctgcgccaaccccatcctctatggctttctctcagacaacttcaagcgctctttccaacgcatcctatgcctcagctggatggacaacgccgcggaggagccggttgactattacgccaccgcgctcaagagccgtgcctacagtgtggaagacttccaacctgagaacctggagtccggcggcgtcttccgtaatggcacctgcacgtcccggatcacgacgctctgagcccgggccacgcaggggctctgagcccgggccacgcaggggccctgagccaaaagagggggagaatgagaagggaaggccgggtgcgaaagggacggtatccagggcgccagggtgctgtcgggataacgtggggctaggacactgacagcctttgatggaggaacccaagaaaggcgcgcgacaatggtagaagtgagagctttgcttataaactgggaaggctttcaggctacctttttctgggtctcccactttctgttccttcctccactgcgcttactcctctgaccctccttctattttccctaccctgcaacttctatcctttcttccgcaccgtcccgccagtgcagatcacgaactcattaacaactcattctgatcctcagcccctccagtcgttatttctgtttgtttaagctgagccacggataccgccacgggtttccctcggcgttagtccctagccgcgcggggccgctgtccaggttctgtctggtgcccctactggagtcccgggaatgaccgctctccctttgcgcagccctaccttaaggaaagttggacttgagaaagatctaagcagctggtcttttctcctactcttgggtgaaggtgcatctttccctgccctcccctgtccccctctcgccgcccgcccgccaccaccactctcactccacccagagtagagccaggtgcttagtaaaataggtcccgcgcttcgaactccaggctttctggagttcccacccaagccctcctttggagcaaagaaggagctgagaacaagccgaatgaggagtttttataagattgcggggtcggagtgtgggcgcgtaataggaatcaccctcctactgcgcgttttcaaagaccaagcgctgggcgctcccgggccgcgcgtctgcgttaggcagggcagggtagtgcagggcacaccttccccggggttcggggttcggggttcggttgcagggctgcagcccgccttggctttctccctcacccaagtttccggaggagccgacctaaaagtaacaatagataaggtttcctgctccagtgtatctcaaaagaccgggcgccaggggcgggggacctagggcgacgtcttcagagtccgccagtgttggcggtgtcgccgcaacctgcaggctcccgagtggggcctgcctggtctctagagggttgctgcctttcaagcggtgcctaagaagttattttcttgtttaacatatatatttattaatttatttgtcgtgttggaaaatgtgtctctgctttccttttctctgcttgcctagccccaggtcttttctttgggaccctgggggcgggcatggaagtggaagtaggggcaagctcttgccccactccctggccatctcaacgcctctcctcaatgctgggccctcttatctcatcctttcctctagcttttctatttttgattgtgttgagtgaagtttggagatttttcatacttttcttactatagtctcttgtttgtcttattaggataatacataaatgataatgtgggttatcctcctctccatgcacagtggaaagtcctgaactcctggctttccaggagacatatataggggaacatcaccctatatataatttgagtgtatatatatttatatatatgatgtggacatatgtatacttatcttgctccattgtcatgagtccatgagtctaagtatagccactgatggtgacaggtgtgagtctggctggaacactttcagtttcaggagtgcaagcagcactcaaacctggagctgaggaatctaattcagacagagactttaatcactgctgaagatgcccctgctccctctgggttccagcagaggtgattcttacatatgatccagttaacatcatcactttttttgaggacattgaaagtgaaataatttgtgtctgtgtttaatattaccaactacattggaagcctgagcagggcgaggaccaataattttaattatttatatttcctgtattgctttagtatgctggcttgtacatagtaggcactaaatacatgtttgttggttgattgtttaagccagagtgtattacaacaatctggagatactaaatctggggttctcaggttcactcattgacatgatatacaatggttaaaatcactattgaaaaatacgttttgtgtatatttgcttcaacaactttgtgctttcctgaaagcagtaaccaagagttaagatatccctaatgttttgcttaaactaatgaacaaatatgctttgggtcataaatcagaaagtttagatctgtcccttaataaaaatatatattactactcctttggaaaatagatttttaatggttaagaactgtgaaatttacaaatcaaaatcttaatcattatccttctaagaggatacaaatttagtgctcttaacttgttaccattgtaatattaactaaataaacagatgtattatgctgttaaaaaaaaaaaaaaaaaaaaaaaaaaaaaaaaaaaaaaaaaaaaaaaaaaaa

SEQ ID NO: 8 - protein, 403 aa, Homo sapiens
MDRSKENCISGPVKATAPVGGPKRVLVTQQFPCQNPLPVNSGQAQRVLCPSNSSQRIPLQAQKLVSSHKPVQNQKQKQLQATSVPHPVSRPLNNTQKSKQPLPSAPENNPEEELASKQKNEESKKRQWALEDFEIGRPLGKGKFGNVYLAREKQSKFILALKVLFKAQLEKAGVEHQLRREVEIQSHLRHPNILRLYGYFHDATRVYLILEYAPLGTVYRELQKLSKFDEQRTATYITELANALSYCHSKRVIHRDIKPENLLLGSAGELKIADFGWSVHAPSSRRTTLCGTLDYLPPEMIEGRMHDEKVDLWSLGVLCYEFLVGKPPFEANTYQETYKRISRVEFTFPDFVTEGARDLISRLLKHNPSQRPMLREVLEHPWITANSSKPSNCQNKESASKQS

SEQ ID NO: 9 - protein, 2549 aa, Homo sapiens
MLGTGPAAATTAATTSSNVSVLQQFASGLKSRNEETRAKAAKELQHYVTMELREMSQEESTRFYDQLNHHIFELVSSSDANERKGGILAIASLIGVEGGNATRIGRFANYLRNLLPSNDPVVMEMASKAIGRLAMAGDTFTAEYVEFEVKRALEWLGADRNEGRRHAAVLVLRELAISVPTFFFQQVQPFFDNIFVAVWDPKQAIREGAVAALRACLILTTQREPKEMQKPQWYRHTFEEAEKGFDETLAKEKGMNRDDRIHGALLILNELVRISSMEGERLREEMEEITQQQLVHDKYCKDLMGFGTKPRHITPFTSFQAVQPQQSNALVGLLGYSSHQGLMGFGTSPSPAKSTLVESRCCRDLMEEKFDQVCQWVLKCRNSKNSLIQMTILNLLPRLAAFRPSAFTDTQYLQDTMNHVLSCVKKEKERTAAFQALGLLSVAVRSEFKVYLPRVLDIIRAALPPKDFAHKRQKAMQVDATVFTCISMLARAMGPGIQQDIKELLEPMLAVGLSPALTAVLYDLSRQIPQLKKDIQDGLLKMLSLVLMHKPLRHPGMPKGLAHQLASPGLTTLPEASDVGSITLALRTLGSFEFEGHSLTQFVRHCADHFLNSEHKEIRMEAARTCSRLLTPSIHLISGHAHVVSQTAVQVVADVLSKLLVVGITDPDPDIRYCVLASLDERFDAHLAQAENLQALFVALNDQVFEIRELAICTVGRLSSMNPAFVMPFLRKMLIQILTELEHSGIGRIKEQSARMLGHLVSNAPRLIRPYMEPILKALILKLKDPDPDPNPGVINNVLATIGELAQVSGLEMRKWVDELFIIIMDMLQDSSLLAKRQVALWTLGQLVASTGYVVEPYRKYPTLLEVLLNFLKTEQNQGTRREAIRVLGLLGALDPYKHKVNIGMIDQSRDASAVSLSESKSSQDSSDYSTSEMLVNMGNLPLDEFYPAVSMVALMRIFRDQSLSHHHTMVVQAITFIFKSLGLKCVQFLPQVMPTFLNVIRVCDGAIREFLFQQLGMLVSFVKSHIRPYMDEIVTLMREFWVMNTSIQSTIILLIEQIVVALGGEFKLYLPQLIPHMLRVFMHDNSPGRIVSIKLLAAIQLFGANLDDYLHLLLPPIVKLFDAPEAPLPSRKAALETVDRLTESLDFTDYASRIIHPIVRTLDQSPELRSTAMDTLSSLVFQLGKKYQIFIPMVNKVLVRHRINHQRYDVLICRIVKGYTLADEEEDPLIYQHRMLRSGQGDALASGPVETGPMKKLHVSTINLQKAWGAARRVSKDDWLEWLRRLSLELLKDSSSPSLRSCWALAQAYNPMARDLFNAAFVSCWSELNEDQQDELIRSIELALTSQDIAEVTQTLLNLAEFMEHSDKGPLPLRDDNGIVLLGERAAKCRAYAKALHYKELEFQKGPTPAILESLISINNKLQQPEAAAGVLEYAMKHFGELEIQATWYEKLHEWEDALVAYDKKMDTNKDDPELMLGRMRCLEALGEWGQLHQQCCEKWTLVNDETQAKMARMAAAAAWGLGQWDSMEEYTCMIPRDTHDGAFYRAVLALHQDLFSLAQQCIDKARDLLDAELTAMAGESYSRAYGAMVSCHMLSELEEVIQYKLVPERREIIRQIWWERLQGCQRIVEDWQKILMVRSLVVSPHEDMRTWLKYASLCGKSGRLALAHKTLVLLLGVDPSRQLDHPLPTVHPQVTYAYMKNMWKSARKIDAFQHMQHFVQTMQQQAQHAIATEDQQHKQELHKLMARCFLKLGEWQLNLQGINESTIPKVLQYYSAATEHDRSWYKAWHAWAVMNFEAVLHYKHQNQARDEKKKLRHASGANITNATTAATTAATATTTASTEGSNSESEAESTENSPTPSPLQKKVTEDLSKTLLMYTVPAVQGFFRSISLSRGNNLQDTLRVLTLWFDYGHWPDVNEALVEGVKAIQIDTWLQVIPQLIARIDTPRPLVGRLIHQLLTDIGRYHPQALIYPLTVASKSTTTARHNAANKILKNMCEHSNTLVQQAMMVSEELIRVAILWHEMWHEGLEEASRLYFGERNVKGMFEVLEPLHAMMERGPQTLKETSFNQAYGRDLMEAQEWCRKYMKSGNVKDLTQAWDLYYHVFRRISKQLPQLTSLELQYVSPKLLMCRDLELAVPGTYDPNQPIIRIQSIAPSLQVITSKQRPRKLTLMGSNGHEFVFLLKGHEDLRQDERVMQLFGLVNTLLANDPTSLRKNLSIQRYAVIPLSTNSGLIGWVPHCDTLHALIRDYREKKKILLNIEHRIMLRMAPDYDHLTLMQKVEVFEHAVNNTAGDDLAKLLWLKSPSSEVWFDRRTNYTRSLAVMSMVGYILGLGDRHPSNLMLDRLSGKILHIDFGDCFEVAMTREKFPEKIPFRLTRMLTNAMEVTGLDGNYRITCHTVMEVLREHKDSVMAVLEAFVYDPLLNWRLMDTNTKGNKRSRTRTDSYSAGQSVEILDGVELGEPAHKKTGTTVPESIHSFIGDGLVKPEALNKKAIQIINRVRDKLTGRDFSHDDTLDVPTQVELLIKQATSHENLCQCYIGWCPFW

SEQ ID NO: 10 - protein, 360 aa, Homo sapiens [sequence omitted]

SEQ ID NO: 11 - protein, 393 aa, Homo sapiens [sequence omitted]

SEQ ID NO: 12 - protein, 249 aa, Homo sapiens
MKLNISFPATGCQKLIEVDDERKLRTFYEKRMATEVAADALGEEWKGYVVRISGGNDKQGFPMKQGVLTHGRVRLLLSKGHSCYRPRRTGERKRKSVRGCIVDANLSVLNLVIVKKGEKDIPGLTDTTVPRRLGPKRASRIRKLFNLSKEDDVRQYVVRKPLNKEGKKPRTKAPKIQRLVTPRVLQHKRRRIALKKQRTKKNKEEAAEYAKLLAKRMKEAKEKRQEQIAKRRRLSSLRASTSKSESSQK

SEQ ID NO: 13 - protein, 480 aa, Homo sapiens [sequence omitted]

SEQ ID NO: 14 - protein, 391 aa, Homo sapiens
MFPNGTASSPSSSPSPSPGSCGEGGGSRGPGAGAADGMEEPGRNASQNGTLSEGQGSAILISFIYSVVCLVGLCGNSMVIYVILRYAKMKTATNIYILNLAIADELLMLSVPFLVTSTLLRHWPFGALLCRLVLSVDAVNMFTSIYCLTVLSVDRYVAVVHPIKAARYRRPTVAKVVNLGVWVLSLLVILPIVVFSRTAANSDGTVACNMLMPEPAQRWLVGFVLYTFLMGFLLPVGAICLCYVLIIAKMRMVALKAGWQQRKRSERKITLMVMMVVMVFVICWMPFYVVQLVNVFAEQDDATVSQLSVILGYANSCANPILYGFLSDNFKRSFQRILCLSWMDNAAEEPVDYYATALKSRAYSVEDFQPENLESGGVFRNGTCTSRITTL

SEQ ID NO: 15 - DNA, 1852 bp, Homo sapiens
accgccgagaccgcgtccgccccgcgagcacagagcctcgcctttgccgatccgccgcccgtccacacccgccgccagctcaccatggatgatgatatcgccgcgctcgtcgtcgacaacggctccggcatgtgcaaggccggcttcgcgggcgacgatgccccccgggccgtcttcccctccatcgtggggcgccccaggcaccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttctaggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcgttgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttcccccttttttgtcccccaacttgagatgtatgaaggcttttggtctccctgggagtgggtggaggcagccagggcttacctgtacactgacttgagaccagttgaataaaagtgcacaccttaaaaatgaaaaaaaaaaaaaaaaaaaaaaaaaaaaaaaaaaaaaaaaa

SEQ ID NO: 16 - DNA, 1310 bp, Homo sapiens
aaattgagcccgcagcctcccgcttcgctctctgctcctcctgttcgacagtcagccgcatcttcttttgcgtcgccagccgagccacatcgctcagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaacc

SEQ ID NO: 17 - DNA, 2245 bp, Homo sapiens
acgcgacccgccctacgggcacctcccgcgcttttcttagcgccgcagacggtggccgagcgggggaccgggaagcatggcccgggggtcggcggttgcctgggcggcgctcgggccgttgttgtggggctgcgcgctggggctgcagggcgggatgctgtacccccaggagagcccgtcgcgggagtgcaaggagctggacggcctctggagcttccgcgccgacttctctgacaaccgacgccggggcttcgaggagcagtggtaccggcggccgctgtgggagtcaggccccaccgtggacatgccagttccctccagcttcaatgacatcagccaggactggcgtctgcggcattttgtcggctgggtgtggtacgaacgggaggtgatcctgccggagcgatggacccaggacctgcgcacaagagtggtgctgaggattggcagtgcccattcctatgccatcgtgtgggtgaatggggtcgacacgctagagcatgaggggggctacctccccttcgaggccgacatcagcaacctggtccaggtggggcccctgccctcccggctccgaatcactatcgccatcaacaacacactcacccccaccaccctgccaccagggaccatccaatacctgactgacacctccaagtatcccaagggttactttgtccagaacacatattttgactttttcaactacgctggactgcagcggtctgtacttctgtacacgacacccaccacctacatcgatgacatcaccgtcaccaccagcgtggagcaagacagtgggctggtgaattaccagatctctgtcaagggcagtaacctgttcaagttggaagtgcgtcttttggatgcagaaaacaaagtcgtggcgaatgggactgggacccagggccaacttaaggtgccaggtgtcagcctctggtggccgtacctgatgcacgaacgccctgcctatctgtattcattggaggtgcagctgactgcacagacgtcactggggcctgtgtctgacttctacacactccctgtggggatccgcactgtggctgtcaccaagagccagttcctcatcaatgggaaacctttctatttccacggtgtcaacaagcatgaggatgcggacatccgagggaagggcttcgactggccgctgctggtgaaggacttcaacctgcttcgctggcttggtgccaacgctttccgtaccagccactacccctatgcagaggaagtgatgcagatgtgtgaccgctatgggattgtggtcatcgatgagtgtcccggcgtgggcctggcgctgccgcagttcttcaacaacgtttctctgcatcaccacatgcaggtgatggaagaagtggtgcgtagggacaagaaccaccccgcggtcgtgatgtggtctgtggccaacgagcctgcgtcccacctagaatctgctggctactacttgaagatggtgatcgctcacaccaaatccttggacccctcccggcctgtgacctttgtgagcaactctaactatgcagcagacaagggggctccgtatgtggatgtgatctgtttgaacagctactactcttggtatcacgactacgggcacctggagttgattcagctgcagctggccacccagtttgagaactggtataagaagtatcagaagcccattattcagagcgagtatggagcagaaacgattgcagggtttcaccaggatccacctctgatgttcactgaagagtaccagaaaagtctgctagagcagtaccatctgggtctggatcaaaaacgcagaaaatacgtggttggagagctcatttggaattttgccgatttcatgactgaacagtcaccgacgagagtgctggggaataaaaaggggatcttcactcggcagagacaaccaaaaagtgcagcgttccttttgcgagagagatactggaagattgccaatgaaaccaggtatccccactcagtagccaagtcacaatgtttggaaaacagcccgtttacttgagcaagactgataccacctgcgtgtcccttcctccccgagtcagggcgacttccacagcagcagaacaagtgcctcctggactgttcacggcagaccagaacgtttctggcctgggttttgtggtcatctattctagcagggaacactaaaggtggaaataaaagattttctattatggaaataaagagttggcatgaaagtggctactgaaaa

SEQ ID NO: 18 - DNA, 1229 bp, Homo sapiens
gtctgacgggcgatggcgcagccaatagacaggagcgctatccgcggtttctgattggctactttgttcgcattataaaaggcacgcgcgggcgcgaggcccttctctcgccaggcgtcctcgtggaagtgacatcgtctttaaaccctgcgtggcaatccctgacgcaccgccgtgatgcccagggaagacagggcgacctggaagtccaactacttccttaagatcatccaactattggatgattatccgaaatgtttcattgtgggagcagacaatgtgggctccaagcagatgcagcagatccgcatgtcccttcgcgggaaggctgtggtgctgatgggcaagaacaccatgatgcgcaaggccatccgagggcacctggaaaacaacccagctctggagaaactgctgcctcatatccgggggaatgtgggctttgtgttcaccaaggaggacctcactgagatcagggacatgttgctggccaataaggtgccagctgctgcccgtgctggtgccattgccccatgtgaagtcactgtgccagcccagaacactggtctcgggcccgagaagacctcctttttccaggctttaggtatcaccactaaaatctccaggggcaccattgaaatcctgagtgatgtgcagctgatcaagactggagacaaagtgggagccagcgaagccacgctgctgaacatgctcaacatctcccccttctcctttgggctggtcatccagcaggtgttcgacaatggcagcatctacaaccctgaagtgcttgatatcacagaggaaactctgcattctcgcttcctggagggtgtccgcaatgttgccagtgtctgtctgcagattggctacccaactgttgcatcagtaccccattctatcatcaacgggtacaaacgagtcctggccttgtctgtggagacggattacaccttcccacttgctgaaaaggtcaaggccttcttggctgatccatctgcctttgtggctgctgcccctgtggctgctgccaccacagctgctcctgctgctgctgcagccccagctaaggttgaagccaaggaagagtcggaggagtcggacgaggatatgggatttggtctctttgactaatcaccaaaaagcaaccaacttagccagttttatttgcaaaacaaggaaataaaggcttacttctttaaaaagtaaaaaaaaaaaaaaaaaaaaaaaaa

SEQ ID NO: 19 - DNA, 5010 bp, Homo sapiens
ggcggctcgggacggaggacgcgctagtgtgagtgcgggcttctagaactacaccgaccctcgtgtcctcccttcatcctgcggggctggctggagcggccgctccggtgctgtccagcagccatagggagccgcacggggagcgggaaagcggtcgcggccccaggcggggcggccgggatggagcggggccgcgagcctgtggggaaggggctgtggcggcgcctcgagcggctgcaggttcttctgtgtggcagttcagaatgatggatcaagctagatcagcattctctaacttgtttggtggagaaccattgtcatatacccggttcagcctggctcggcaagtagatggcgataacagtcatgtggagatgaaacttgctgtagatgaagaagaaaatgctgacaataacacaaaggccaatgtcacaaaaccaaaaaggtgtagtggaagtatctgctatgggactattgctgtgatcgtctttttcttgattggatttatgattggctacttgggctattgtaaaggggtagaaccaaaaactgagtgtgagagactggcaggaaccgagtctccagtgagggaggagccaggagaggacttccctgcagcacgtcgcttatattgggatgacctgaagagaaagttgtcggagaaactggacagcacagacttcaccagcaccatcaagctgctgaatgaaaattcatatgtccctcgtgaggctggatctcaaaaagatgaaaatcttgcgttgtatgttgaaaatcaatttcgtgaatttaaactcagcaaagtctggcgtgatcaacattttgttaagattcaggtcaaagacagcgctcaaaactcggtgatcatagttgataagaacggtagacttgtttacctggtggagaatcctgggggttatgtggcgtatagtaaggctgcaacagttactggtaaactggtccatgctaattttggtactaaaaaagattttgaggatttatacactcctgtgaatggatctatagtgattgtcagagcagggaaaatcacctttgcagaaaaggttgcaaatgctgaaagcttaaatgcaattggtgtgttgatatacatggaccagactaaatttcccattgttaacgcagaactttcattctttggacatgctcatctggggacaggtgacccttacacacctggattcccttccttcaatcacactcagtttccaccatctcggtcatcaggattgcctaatatacctgtccagacaatctccagagctgctgcagaaaagctgtttgggaatatggaaggagactgtccctctgactggaaaacagactctacatgtaggatggtaacctcagaaagcaagaatgtgaagctcactgtgagcaatgtgctgaaagagataaaaattcttaacatctttggagttattaaaggctttgtagaaccagatcactatgttgtagttggggcccagagagatgcatggggccctggagctgcaaaatccggtgtaggcacagctctcctattgaaacttgcccagatgttctcagatatggtcttaaaagatgggtttcagcccagcagaagcattatctttgccagttggagtgctggagactttggatcggttggtgccactgaatggctagagggatacctttcgtccctgcatttaaaggctttcacttatattaatctggataaagcggttcttggtaccagcaacttcaaggtttctgccagcccactgttgtatacgcttattgagaaaacaatgcaaaatgtgaagcatccggttactgggcaatttctatatcaggacagcaactgggccagcaaagttgagaaactcactttagacaatgctgctttccctttccttgcatattctggaatcccagcagtttctttctgtttttgcgaggacacagattatccttatttgggtaccaccatggacacctataaggaactgattgagaggattcctgagttgaacaaagtggcacgagcagctgcagaggtcgctggtcagttcgtgattaaactaacccatgatgttgaattgaacctggactatgagaggtacaacagccaactgctttcatttgtgagggatctgaaccaatacagagcagacataaaggaaatgggcctgagtttacagtggctgtattctgctcgtggagacttcttccgtgctacttccagactaacaacagatttcgggaatgctgagaaaacagacagatttgtcatgaagaaactcaatgatcgtgtcatgagagtggagtatcacttcctctctccctacgtatctccaaaagagtctcctttccgacatgtcttctggggctccggctctcacacgctgccagctttactggagaacttgaaactgcgtaaacaaaataacggtgcttttaatgaaacgctgttcagaaaccagttggctctagctacttggactattcagggagctgcaaatgccctctctggtgacgtttgggacattgacaatgagttttaaatgtgatacccatagcttccatgagaacagcagggtagtctggtttctagacttgtgctgatcgtgctaaattttcagtagggctacaaaacctgatgttaaaattccatcccatcatcttggtactactagatgtctttaggcagcagcttttaatacagggtagataacctgtacttcaagttaaagtgaataaccacttaaaaaatgtccatgatggaatattcccctatctctagaattttaagtgctttgtaatgggaactgcctctttcctgttgttgttaatgaaaatgtcagaaaccagttatgtgaatgatctctctgaatcctaagggctggtctctgctgaaggttgtaagtggttcgcttactttgagtgatcctccaacttcatttgatgctaaataggagataccaggttgaaagacctctccaaatgagatctaagcctttccataaggaatgtagcaggtttcctcattcctgaaagaaacagttaactttcagaagagatgggcttgttttcttgccaatgaggtctgaaatggaggtccttctgctggataaaatgaggttcaactgttgattgcaggaataaggccttaatatgttaacctcagtgtcatttatgaaaagaggggaccagaagccaaagacttagtatattttcttttcctctgtcccttcccccataagcctccatttagttctttgttatttttgtttcttccaaagcacattgaaagagaaccagtttcaggtgtttagttgcagactcagtttgtcagactttaaagaataatatgctgccaaattttggccaaagtgttaatcttaggggagagctttctgtccttttggcactgagatatttattgtttatttatcagtgacagagttcactataaatggtgtttttttaatagaatataattatcggaagcagtgccttccataattatgacagttatactgtcggttttttttaaataaaagcagcatctgctaataaaacccaacagatactggaagttttgcatttatggtcaacacttaagggttttagaaaacagccgtcagccaaatgtaattgaataaagttgaagctaagatttagagatgaattaaatttaattaggggttgctaagaagcgagcactgaccagataagaatgctggttttcctaaatgcagtgaattgtgaccaagttataaatcaatgtcacttaaaggctgtggtagtactcctgcaaaattttatagctcagtttatccaaggtgtaactctaattcccatttgcaaaatttccagtacctttgtcacaatcctaacacattatcgggagcagtgtcttccataatgtataaagaacaaggtagtttttacctaccacagtgtctgtatcggagacagtgatctccatatgttacactaagggtgtaagtaattatcgggaacagtgtttcccataattttcttcatgcaatgacatcttcaaagcttgaagatcgttagtatctaacatgtatcccaactcctataattccctatcttttagttttagttgcagaaacattttgtggtcattaagcattgggtgggtaaattcaaccactgtaaaatgaaattactacaaaatttgaaatttagcttgggtttttgttacctttatggtttctccaggtcctctacttaatgagatagcagcatacatttataatgtttgctattgacaagtcattttaatttatcacattatttgcatgttacctcctataaacttagtgcggacaagttttaatccagaattgaccttttgacttaaagcagagggactttgtatagaaggtttgggggctgtggggaaggagagtcccctgaaggtctgacacgtctgcctacccattcgtggtgatcaattaaatgtaggtatgaataagttcgaagctccgtgagtgaaccatcatataaacgtgtagtacagctgtttgtcatagggcagttggaaacggcctcctagggaaaagttcatagggtctcttcaggttcttagtgtcacttacctagatttacagcctcacttgaatgtgtcactactcacagtctctttaatcttcagttttatctttaatctcctcttttatcttggactgacatttagcgtagctaagtgaaaaggtcatagctgagattcctggttcgggtgttacgcacacgtacttaaatgaaagcatgtggcatgttcatcgtataacacaatatgaatacagggcatgcattttgcagcagtgagtctcttcagaaaacccttttctacagttagggttgagttacttcctatcaagccagtacgtgctaacaggctcaatattcctgaatgaaatatcagactagtgacaagctcctggtcttgagatgtcttctcgttaaggagtagggccttttggaggtaaaggtata

SEQ ID NO: 20 - protein, 375 aa, Homo sapiens
MDDDIAALVVDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 21 - protein, 335 aa, Homo sapiens
MGKVKVGVNGFGRIGRLVTRAAFNSGKVDIVAINDPFIDLNYMVYMFQYDSTHGKFHGTVKAENGKLVINGNPITIFQERDPSKIKWGDAGAEYVVESTGVFTTMEKAGAHLQGGAKRVIISAPSADAPMFVMGVNHEKYDNSLKIISNASCTTNCLAPLAKVIHDNFGIVEGLMTTVHAITATQKTVDGPSGKLWRDGRGALQNIIPASTGAAKAVGKVIPELNGKLTGMAFRVPTANVSVVDLTCRLEKPAKYDDIKKVVKQASEGPLKGILGYTEHQVVSSDFNSDTHSSTFDAGAGIALNDHFVKLISWYDNEFGYSNRVVDLMAHMASKE

SEQ ID NO: 22 - protein, 651 aa, Homo sapiens
MARGSAVAWAALGPLLWGCALGLQGGMLYPQESPSRECKELDGLWSFRADFSDNRRRGFEEQWYRRPLWESGPTVDMPVPSSFNDISQDWRLRHFVGWVWYEREVILPERWTQDLRTRVVLRIGSAHSYAIVWVNGVDTLEHEGGYLPFEADISNLVQVGPLPSRLRITIAINNTLTPTTLPPGTIQYLTDTSKYPKGYFVQNTYFDFFNYAGLQRSVLLYTTPTTYIDDITVTTSVEQDSGLVNYQISVKGSNLFKLEVRLLDAENKVVANGTGTQGQLKVPGVSLWWPYLMHERPAYLYSLEVQLTAQTSLGPVSDFYTLPVGIRTVAVTKSQFLINGKPFYFHGVNKHEDADIRGKGFDWPLLVKDFNLLRWLGANAFRTSHYPYAEEVMQMCDRYGIVVIDECPGVGLALPQFFNNVSLHHHMQVMEEVVRRDKNHPAVVMWSVANEPASHLESAGYYLKMVIAHTKSLDPSRPVTFVSNSNYAADKGAPYVDVICLNSYYSWYHDYGHLELIQLQLATQFENWYKKYQKPIIQSEYGAETIAGFHQDPPLMFTEEYQKSLLEQYHLGLDQKRRKYVVGELIWNFADFMTEQSPTRVLGNKKGIFTRQRQPKSAAFLLRERYWKIANETRYPHSVAKSQCLENSPFT

SEQ ID NO: 23 - protein, 317 aa, Homo sapiens
MPREDRATWKSNYFLKIIQLLDDYPKCFIVGADNVGSKQMQQIRMSLRGKAVVLMGKNTMMRKAIRGHLENNPALEKLLPHIRGNVGFVFTKEDLTEIRDMLLANKVPAAARAGAIAPCEVTVPAQNTGLGPEKTSFFQALGITTKISRGTIEILSDVQLIKTGDKVGASEATLLNMLNISPFSFGLVIQQVFDNGSIYNPEVLDITEETLHSRFLEGVRNVASVCLQIGYPTVASVPHSIINGYKRVLALSVETDYTFPLAEKVKAFLADPSAFVAAAPVAAATTAAPAAAAAPAKVEAKEESEESDEDMGFGLFD

SEQ ID NO: 24 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTSTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

(19) United States
(12) Patent Application Publication
(10) Pub. No.: US 2011/0059464 A1
(43) Pub. Date: Mar. 10, 2011

(54) Biomarker Panel For Prediction Of Recurrent Colorectal Cancer

(76) Inventor: Patrick J. Muraca, Pittsfield, MA (US)

(21) Appl. No.: 12/936,231

(22) PCT Filed: Apr. 6, 2009
(86) PCT No.: PCT/US09/39575

§ 371 Date: Nov. 23, 2010

Related U.S. Application Data
(60) Provisional application No. 61/123,376, filed on Apr. 8, 2008.

(51) Int. Cl. G01N 33/53; C12Q 1/02

(52) U.S. Cl. 435/7.1; 435/29

(57) ABSTRACT

The present invention provides a biomarker panel predictive of whether colorectal cancer is likely to recur or metastasize in an afflicted patient. By identifying the likelihood of recurrence, a treatment provider may determine in advance those patients who would benefit from certain types of treatment. The present invention further provide methods of identifying gene and protein expression profiles associated with the likelihood of recurrence/metastasis of colorectal cancer in a patient sample.

### RELATED APPLICATIONS

This application claims priority under 35 U.S.C. §119(e) to U.S. provisional Application Ser. No. 61/123,376, filed Apr. 8, 2008, the entirety of which is incorporated herein by reference.

### BACKGROUND OF THE INVENTION

Treatment of recurrent colon cancer depends on the sites of recurrent disease demonstrable by physical examination and/or radiographic studies. In addition to standard radiographic procedures, radioimmunoscintography may add clinical information which may affect management. Serafini, et al., “Radioimmunoscintigraphy of recurrent, metastatic, or occult colorectal cancer with technetium 99m-labled totally human monoclonal antibody 88BV59: results of pivotal, phase III multicenter studies.” Journal of Clinical Oncology, 16(5): 1777-1787 (1998). However, such approaches have not led to improvements in long-term outcome measures such as survival.

Recurrence of colon cancer often occurs at sites and in tissues other than the site of the primary tumor (referred to as metastasis). Treatments of liver metastases of colorectal cancer include resection of metastases, cryotherapy, and/or intra-arterial chemotherapy using improved implantable infusion ports and pumps. Kemen, et al., “Randomized trial of hepatic arterial floxuridine, mitomycin, and carmustine versus floxuridine alone in previously treated patients with liver metastases from colorectal cancer.” Journal of Clinical Oncology, 11(2): 330-335, (1993); Pedersen et al., “Resection of liver metastases from colorectal cancer: indications and results.” Diseases of the Colon and Rectum, 37(11): 1078-1082 (1994); Korpan, “Hepatic cryosurgery for liver metastases: long-term follow-up.” Annals of Surgery, 225(2): 193-201 (1997); Adam R, Akpinar, et al., “Place of cryosurgery in the treatment of malignant liver tumors. Annals of Surgery, 225(1): 39-50 (1997). For those patients with hepatic metastases deemed unresectable, cryosurgical ablation has been associated with long term tumor control. Prognostic variables that predict a favorable outcome for cryotherapy are similar to those for hepatic resection and include low preoperative carcinoembryonic antigen level, absence of extrahepatic disease, negative margin, and lymph node negative primary. Seifert, et al., “Prognostic factors after cryotherapy for hepatic metastases from colorectal cancer.” Annals of Surgery, 228(2): 201-208 (1998).

Locally recurrent colon cancer, such as a suture line recurrence, may be resectable, particularly if an inadequate prior operation was performed. Limited pulmonary metastases may also be considered for surgical resection, with 5-year survival possible in highly selected patients. McAfee, et al., “Colorectal lung metastases: results of surgical excision.” Annals of Thoracic Surgery, 53(5): 780-786 (1992); Girard, et al., “Surgery for lung metastases from colorectal cancer: analysis of prognostic factors.” Journal of Clinical Oncology, 14(7): 2047-2053 (1996).

In stage 1V and recurrent colon cancer, chemotherapy has been used for palliation, with fluorouracil (5-FU)-based treatment considered to be standard. Moertel, “Chemotherapy for colorectal cancer.” New England Journal of Medicine, 330(16): 1136-1142 (1994). Combination chemotherapy has not been shown to be more effective than 5-FU alone. 5-FU has been shown to be more cytotoxic, with increased response rates but with variable effects on survival, when modulated by leucovorin, methotrexate, or other agents. Valone, et al., “Treatment of patients with advanced colorectal carcinomas with fluorouracil alone, high-dose leucovorin plus fluorouracil, or sequential methotrexate, fluorouracil, and leucovorin: a randomized trial of the Northern California Oncology Group.” Journal of Clinical Oncology, 7(10): 1427-1436 (1989); Jager, et al, “Weekly high-dose leucovorin versus low-dose leucovorin combined with fluorouracil in advanced colorectal cancer: results of a randomized multicenter trial.” Journal of Clinical Oncology, 14(8): 2274-2279 (1996); The Advanced Colorectal Cancer Meta-Analysis Project: Meta-analysis of randomized trials testing the biochemical modulation of fluorouracil by methotrexate in metastatic colorectal cancer. Journal of Clinical Oncology, 12(5): 960-969 (1994).

Interferon alfa appears to add toxic effects but no clinical benefit to 5-FU therapy. Kosmidis, et al., “Fluorouracil and leucovorin with or without interferon alfa-2b in advanced colorectal cancer: analysis of a prospective randomized phase III trial.” Journal of Clinical Oncology, 14(10): 2682-2687 (1996); Greco, et al., “Phase III randomized study to compare interferon alfa-2a in combination with fluorouracil versus fluorouracil alone in patients with advanced colorectal cancer.” Journal of Clinical Oncology, 14(10): 2674-2681 (1996). Continuous-infusion 5-FU regimens have also resulted in increased response rates in some studies, with a modest benefit in median survival. Hansen, et al. “Phase III study of bolus versus infusion fluorouracil with or without cisplatin in advanced colorectal cancer.” Journal of the National Cancer Institute, 88(10): 668-674 (1996); Aranda, et al., “Randomized trial comparing monthly low-dose leucovorin and fluorouracil bolus with weekly high-dose 48-hour continuous infusion fluorouracil for advanced colorectal cancer: a Spanish Cooperative Group for Gastrointestinal Tumor Therapy (TTD) study.” Annals of Oncology, 9(7): 727-731 (1998). The choice of a 5-FU-based chemotherapy regimen for an individual patient should be based on known response rates and the toxic effects profile of the chosen regimen, as well as cost and quality-of-life issues. Leichman, et al., “Phase II study of fluorouracil and its modulation in advanced colorectal cancer: a Southwest Oncology Group study.” Journal of Clinical Oncology, 13(6): 1303-1311 (1995).

Irinotecan is a topoisomerase-I inhibitor with a 10% to 20% partial response rate in patients with metastatic colon cancer, in patients who have received no prior chemotherapy, and in patients progressing on 5-FU therapy. It is now considered standard therapy for patients with stage 1V disease who do not respond to or progress on 5-FU. Cunningham, et al. “A phase III multicenter randomized study of CPT-11 versus supportive care (SC) alone in patients (Pts) with 5FU-resistant metastatic colorectal cancer (MCRC).”Proceedings of the American Society of Clinical Oncology, 17: A-1, 1a (1998). Another drug, Tomudex, is a specific thymidylate synthase inhibitor which has demonstrated activity similar to that of bolus 5-FU and leucovorin. Cunningham D, “Mature results from three large controlled studies with raltitrexed (‘Tomudex’).” British Journal of Cancer, 77(Suppl 2): 15-21 (1998); Cocconi, et al., “Open, randomized, multicenter trial of raltitrexed versus fluorouracil plus high-dose leucovorin in patients with advanced colorectal cancer.” Journal of Clinical Oncology, 16(9): 2943-2952, (1998). Oxaliplatin plus 5-FU and leucovorin has also shown activity in 5-FU refractory patients. Von Hoff DD, “Promising new agents for treatment of patients with colorectal cancer. Seminars in Oncology, 25(5, suppl 11): 47-52 (1998); de Gramont, et al., “Oxaliplatin with high-dose leucovorin and 5-fluorouracil 48-hour continuous infusion in pretreated metastatic colorectal cancer.” European Journal of Cancer, 33(2): 214-219 (1997).

Patients with advanced colon cancer who have relapsed after either adjuvant therapy or treatment for advanced disease with 5-FU and leucovorin may be considered for additional therapy. A number of approaches have been used in the treatment of such patients, including retreatment with 5-FU and treatment with irinotecan. Patients retreated with bolus or infusional 5-FU following adjuvant 5-FU therapy or discontinuation of 5-FU in responding patients with metastatic disease have response rates and response durations similar to previously untreated patients. Goldberg R M, “Is repeated treatment with a 5-fluorouracil-based regimen useful in colorectal cancer?” Seminars in Oncology, 25(5, suppl 11): 21-28 (1998). Irinotecan has been compared to either retreatment with 5-FU or best supportive care in a pair of randomized European trials of patients with colorectal cancer refractory to 5-FU. In both trials, there was a survival and quality of life advantage for patients treated with irinotecan over 5-FU or supportive care. Rougier, et al., “Randomised trial of irinotecan versus fluorouracil by continuous infusion after fluorouracil failure in patients with metastatic colorectal cancer.” Lancet, 352(9138): 1407-1412 (1998); Cunningham, et al., “Randomised trial of irinotecan plus supportive care versus supportive care alone after fluorouracil failure for patients with metastatic colorectal cancer.” Lancet, 352(9138): 1413-1418 (1998).

### SUMMARY OF THE INVENTION

The present invention provides gene and protein expression profiles and methods for using them to identify those patients who are likely to experience a recurrence and/or metastasis of their colon cancer after treatment of the primary tumor, as well as those patients that are not likely to experience a recurrence of their cancer. The present invention allows a treatment provider to identify those patients who are most likely to experience recurrence, and to adjust treatment options for such patients accordingly.

In one aspect, the present invention comprises protein expression profiles that are indicative of the likelihood that a colon cancer patient's disease will recur/metastasize. The protein expression profiles comprise proteins that are differentially expressed in colon cancer patients whose disease is unlikely to recur after treatment of the primary tumor. The present protein expression profile (PEP) comprises at least one, and preferably a plurality, of proteins selected from the group consisting of: phospho-AIK, phospho-mTOR, phospho MAPK, phospho-MEK, phospho-S6, AKT, and SSTR1. All of these proteins are up-regulated (overexpressed) in the colon tumors of patients whose colon cancer is are not likely to recur and/or metastasize.

The present invention further comprises gene expression profiles, also referred to as “gene signatures,” that are indicative of the likelihood that a patient's colon cancer will recur/metastasize after treatment of the primary tumor. The gene expression profile (GEP) comprises at least one, and preferably a plurality, of genes selected from the group consisting of genes encoding the following proteins: AIK, mTOR, MAPK, MEK, S6, AKT and SSTR1. These genes are up-regulated (over-expressed) in the tumors of those patients whose cancer is not likely to recur after treatment of the primary tumor.

The present gene and protein expression profiles further may include reference or control genes and the proteins expressed thereby. The currently preferred reference genes are ACTB, GAPD, GUSB, RPLP0 and TFRC. According to the invention, some or all of theses genes and their encoded proteins are differentially expressed (e.g., up-regulated or down-regulated) in patients whose colon cancer is not likely to recur after treatment for the primary tumor. Specifically, all of these genes and their encoded proteins are up-regulated (over-expressed) in patients at low risk of recurrence of their colon cancer after treatment of the primary tumor.

The gene and protein expression profiles of the present invention (referred to hereinafter as GPEPs) comprise a group of genes and proteins that are up-regulated in colon cancer patients whose cancers are unlikely to recur/metastasize after treatment of the primary tumor, relative to expression of the same genes in the primary colon tumors of patients whose cancers are likely to recur/metastasize. The GPEPs of the present invention thus can be used to predict the likelihood of recurrence of the cancer and/or disease-related death. The present GPEP also can be used to identify those colon cancer patients most likely to respond to standard therapy of their primary tumors, as well as those requiring adjuvant therapies.

The present invention further comprises a method of determining if a colon cancer patient's disease is of a type that is likely to recur/metastasize after treatment of the primary tumor. The method comprises obtaining a tumor sample from the patient, determining the gene and/or protein expression profile of the sample, and determining from the gene or protein expression profile whether at least about 2, preferably at least about 4, and most preferably about 7 of the genes that encode the proteins selected from the group consisting of: AIK, mTOR, MAPK, MEK, S6, AKT and SSTR1, or whether at least about 2, preferably at least about 4, and most preferably about 7 proteins selected from the group consisting of: phospho-AIK, phospho-mTOR, phospho MAPK, phospho-MEK, phospho-S6, AKT, and SSTR1, are differentially expressed in the sample. From this information, the treatment provider can ascertain whether the patient's disease is likely to recur and/or metastasize, and tailor the patient's treatment accordingly.

The present invention further comprises assays for determining the gene and/or protein expression profile in a patient's sample, and instructions for using the assay. The assay may be based on detection of nucleic acids (e.g., using nucleic acid probes specific for the nucleic acids of interest) or proteins or peptides (e.g., using antibodies specific for the proteins/peptides of interest). In a currently preferred embodiment, the assay comprises an immunohistochemistry (IHC) test in which tissue samples, preferably from the primary resected tumor, are contacted with antibodies specific for the proteins/peptides identified in the GPEP as being indicative of the likelihood of recurrence/metastasis of colon cancer in the patient after treatment of the primary tumor.

The GPEP, method and assay of the present invention can be used to accurately predict whether a colon cancer patient's disease is likely to recur and/or metastasize. This knowledge allows the patient and caregiver to make better clinical decisions, e.g., frequency of monitoring, administration of adjuvant radiation or chemotherapy, or design of an appropriate therapeutic regimen.

### DETAILED DESCRIPTION

The present invention provides gene and protein expression profiles and their use for predicting the likelihood of recurrence and/or metastasis of colon cancer after treatment of the primary tumor. More specifically, the present GPEPs are indicative of whether colon cancer is likely to recur in the patient's colorectal tissue or metastasize (recur at a different site, such as the liver or lung), after treatment of the primary tumor.

Treatment of recurrent/metastatic colon cancer depends on the sites of recurrent disease. Recurrence currently is determined mainly by physical examination and/or radiographic studies; radioimmunoscintography may add additional clinical information which affects management of the disease. However, these approaches have not led to improvements in long-term outcome measures such as survival. The GPEP of the present invention provides the clinician with a prognostic tool capable of providing valuable information that can positively affect management of the disease. Oncologists can assay the primary tumor for the presence of the present GPEP, and which can identify with a high degree of accuracy those patients whose disease is likely to recur or metastasize. This information, taken together with other available clinical information, allows more effective management of the disease.

In a preferred aspect of the invention, the expression of proteins in a tumor sample from a colon cancer patient is assayed using immunohistochemistry techniques to identify the expression of proteins in the present GPEP. The protein expression profile comprises at least two, preferably a plurality, and most preferably all, of the proteins selected from the group consisting of phospho-AIK, phospho-mTOR, phospho-MAPK, phospho MEK, phospho-S6, AKT and SSTR1. According to the invention, some or all of these proteins are differentially expressed in patients who are least at risk for recurrence/metastasis of their colon cancer. Specifically, these proteins are up-regulated (over-expressed) in patients who are not likely to experience recurrence/metastasis of their disease.

In this embodiment, the method comprises (a) obtaining a biological sample (preferably primary resected tumor) of a patient afflicted with colon cancer; (b) contacting the sample with nucleic acid probes or antibodies specific for the following proteins: phospho-AIK, phospho-mTOR, phospho-MAPK, phospho MEK, phospho-S6, AKT and SSTR1; and (c) determining whether two or more of these proteins are up-regulated (over-expressed). The predictive value of the PEP for determining the likelihood of recurrence increases with the number of these proteins that are found to be up-regulated. Preferably, at least about two, more preferably at least about four, and most preferably about seven, of these proteins in the present GPEP are overexpressed. In a preferred embodiment, samples of normal (undiseased) colon margin tissue (tissue form the patient's colon surrounding the tumor site) as well as other control tissues are assayed simultaneously, using the same reagents and under the same conditions, with the primary tumor sample. Preferably, expression of at least two reference proteins also is measured at the same time and under the same conditions.

In an alternative embodiment, the present invention comprises gene expression profiles that are indicative of the likelihood of recurrence/metastasis of disease in a colon cancer patient. In this embodiment, the present method comprises (a) obtaining a biological sample (preferably primary resected tumor) of a patient afflicted with colon cancer; (b) contacting the sample with nucleic acid probes specific for the following genes: AIK, mTOR, MAPK, MEK, S6, AKT and SSTR1; and (c) determining whether two or more of these genes are up-regulated (over-expressed). The predictive value of the gene profile for determining the likelihood of recurrence increases with the number of these genes that are found to be up-regulated in accordance with the invention. Preferably, at least about two, more preferably at least about four, and most preferably about seven, of the genes in the present GPEP are differentially expressed. The biological sample preferably is a sample of the patient's primary resected tumor; normal (undiseased) marginal colon tissue from the same patient is used as a control. Preferably, expression of at least two reference genes also is measured.

In a currently preferred embodiment, the present gene and protein expression profiles further may include determining the expression levels of reference or control genes and the proteins. The currently preferred reference genes are ACTB, GAPD, GUSB, RPLP0 and TFRC. According to the invention, some or all of theses genes and their encoded proteins are differentially expressed (e.g., up-regulated or down-regulated) in patients whose colon cancer is not likely to recur after treatment for the primary tumor.

The present invention further comprises assays for determining the gene and/or protein expression profile in a patient's sample, and instructions for using the assay. The assay may be based on detection of nucleic acids (e.g., using nucleic acid probes specific for the nucleic acids of interest) or proteins or peptides (e.g., using nucleic acid probes or antibodies specific for the proteins/peptides of interest). In a currently preferred embodiment, the assays comprises an immunohistochemistry (IHC) test in which tissue samples, preferably arrayed in a tissue microarray (TMA), and are contacted with antibodies specific for the proteins/peptides identified in the GPEP as being indicative of the likelihood of recurrence/metastasis of colon cancer in patient after treatment of the primary tumor.

Table 1 identifies the genes and the (unphosphorylated) protein encoded thereby in the present GPEP. Table 1 also indicates whether expression of the gene and protein is up- or down-regulated in patients unlikely to experience recurrence or metastasis of their disease.

Table 2 identifies the five preferred reference genes and the protein encoded thereby. Table 2 also indicates whether expression of the reference gene and protein is up- or down-regulated in patients unlikely to experience recurrence or metastasis of their disease.

Tables 1 and 2 include the NCBI Accession No. of a variant of each gene and protein; other variants of these genes and proteins exist, which can be readily ascertained by reference to an appropriate database such as NCBI Entrez (available via the NIH website). Alternate names for the genes and proteins listed in Table 1 also can be determined from the NCBI site.

TABLE 1

| Gene | SEQ ID NO. | Encoded Protein | SEQ ID NO. |
|---|---|---|---|
| Accession No. | for Gene | Accession No. | for Protein |
| AURKA | 1 | AIK | 8 |
| NM _198433.1 | | NP_940835.1 | |
| FRAP1 | 2 | mTOR | 9 |
| NM_004958.2 | | NP_004949.1 | |
| MAPK1 | 3 | MAPK | 10 |
| NM_002745.4 | | NP_002736.3 | |
| MAP2K1 | 4 | MEK | 11 |
| NM_002755.3 4 | | NP_0002746.1 11 | |
| RPS6 | 5 | S6 | 12 |
| NM_001010.2 5 | | NP_001001.2 12 | |
| AKT | 6 | AKT | 13 |
| NM_005163.2 6 | | NP_005154.2 13 | |
| SSTR1 | 7 | SSTR1 | 14 |
| NM_001049.2 7 | | NP_001040.1 14 | |

TABLE 2

| Gene | SEQ ID NO. | Encoded Protein | SEQ ID NO. |
|---|---|---|---|
| Accession No. | for Gene | Accession No. | for Protein |
| ACTB | 15 | β-Actin | 20 |
| NM_001101.3 | | NP_001092.1 | |
| GAPD | 16 | GAPD | 21 |
| NM_002046.3 | | NP_002037.2 | |
| GUSB | 17 | GUS | 22 |
| NM_000181.2 | | NP_000172.1 | |
| RPLP0 | 18 | Ribosomal protein P0 | 23 |
| NM_001002.3 | | NP_000993.1 | |
| TFRC | 19 | Transferrin receptor | 24 |
| NM_003234.1 | | NP_003225.1 | |

All of the genes and proteins listed in Tables 1 and 2 are up-regulated (overexpressed) in the colon tumors of patients whose colon cancer is are not likely to recur and/or metastasize.

### DEFINITIONS

For convenience, the meaning of certain terms and phrases employed in the specification, examples, and appended claims are provided below. The definitions are not meant to be limiting in nature and serve to provide a clearer understanding of certain aspects of the present invention.

The term “genome” is intended to include the entire DNA complement of an organism, including the nuclear DNA component, chromosomal or extrachromosomal DNA, as well as the cytoplasmic domain (e.g., mitochondrial DNA).

The term “gene” refers to a nucleic acid sequence that comprises control and coding sequences necessary for producing a polypeptide or precursor. The polypeptide may be encoded by a full length coding sequence or by any portion of the coding sequence. The gene may be derived in whole or in part from any source known to the art, including a plant, a fungus, an animal, a bacterial genome or episome, eukaryotic, nuclear or plasmid DNA, cDNA, viral DNA, or chemically synthesized DNA. A gene may contain one or more modifications in either the coding or the untranslated regions that could affect the biological activity or the chemical structure of the expression product, the rate of expression, or the manner of expression control. Such modifications include, but are not limited to, mutations, insertions, deletions, and substitutions of one or more nucleotides. The gene may constitute an uninterrupted coding sequence or it may include one or more introns, bound by the appropriate splice junctions. The Term “gene” as used herein includes variants of the genes identified in Table 1.

The term “gene expression” refers to the process by which a nucleic acid sequence undergoes successful transcription and translation such that detectable levels of the nucleotide sequence are expressed.

The terms “gene expression profile” or “gene signature” refer to a group of genes expressed by a particular cell or tissue type wherein presence of the genes taken together or the differential expression of such genes, is indicative/predictive of a certain condition.

The term “nucleic acid” as used herein, refers to a molecule comprised of one or more nucleotides, i.e., ribonucleotides, deoxyribonucleotides, or both. The term includes monomers and polymers of ribonucleotides and deoxyribonucleotides, with the ribonucleotides and/or deoxyribonucleotides being bound together, in the case of the polymers, via 5′ to 3′ linkages. The ribonucleotide and deoxyribonucleotide polymers may be single or double-stranded. However, linkages may include any of the linkages known in the art including, for example, nucleic acids comprising 5′ to 3′ linkages. The nucleotides may be naturally occurring or may be synthetically produced analogs that are capable of forming base-pair relationships with naturally occurring base pairs. Examples of non-naturally occurring bases that are capable of forming base-pairing relationships include, but are not limited to, aza and deaza pyrimidine analogs, aza and deaza purine analogs, and other heterocyclic base analogs, wherein one or more of the carbon and nitrogen atoms of the pyrimidine rings have been substituted by heteroatoms, e.g., oxygen, sulfur, selenium, phosphorus, and the like. Furthermore, the term “nucleic acid sequences” contemplates the complementary sequence and specifically includes any nucleic acid sequence that is substantially homologous to the both the nucleic acid sequence and its complement.

The terms “array” and “microarray” refer to the type of genes or proteins represented on an array by oligonucleotides or protein-capture agents, and where the type of genes or proteins represented on the array is dependent on the intended purpose of the array (e.g., to monitor expression of human genes or proteins). The oligonucleotides or protein-capture agents on a given array may correspond to the same type, category, or group of genes or proteins. Genes or proteins may be considered to be of the same type if they share some common characteristics such as species of origin (e.g., human, mouse, rat); disease state (e.g., cancer); functions (e.g., protein kinases, tumor suppressors); or same biological process (e.g., apoptosis, signal transduction, cell cycle regulation, proliferation, differentiation). For example, one array type may be a “cancer array” in which each of the array oligonucleotides or protein-capture agents correspond to a gene or protein associated with a cancer. An “epithelial array” may be an array of oligonucleotides or protein-capture agents corresponding to unique epithelial genes or proteins. Similarly, a “cell cycle array” may be an array type in which the oligonucleotides or protein-capture agents correspond to unique genes or proteins associated with the cell cycle.

The term “cell type” refers to a cell from a given source (e.g., a tissue, organ) or a cell in a given state of differentiation, or a cell associated with a given pathology or genetic makeup.

The term “activation” as used herein refers to any alteration of a signaling pathway or biological response including, for example, increases above basal levels, restoration to basal levels from an inhibited state, and stimulation of the pathway above basal levels.

The term “differential expression” refers to both quantitative as well as qualitative differences in the temporal and tissue expression patterns of a gene or a protein in diseased tissues or cells versus normal adjacent tissue. For example, a differentially expressed gene may have its expression activated or completely inactivated in normal versus disease conditions, or may be up-regulated (over-expressed) or down-regulated (under-expressed) in a disease condition versus a normal condition. Such a qualitatively regulated gene may exhibit an expression pattern within a given tissue or cell type that is detectable in either control or disease conditions, but is not detectable in both. Stated another way, a gene or protein is differentially expressed when expression of the gene or protein occurs at a higher or lower level in the diseased tissues or cells of a patient relative to the level of its expression in the normal (disease-free) tissues or cells of the patient and/or control tissues or cells.

The term “detectable” refers to an RNA expression pattern which is detectable via the standard techniques of polymerase chain reaction (PCR), reverse transcriptase-(RT) PCR, differential display, and Northern analyses, which are well known to those of skill in the art. Similarly, protein expression patterns may be “detected” via standard techniques such as Western blots.

The term “complementary” refers to the topological compatibility or matching together of the interacting surfaces of a probe molecule and its target. The target and its probe can be described as complementary, and furthermore, the contact surface characteristics are complementary to each other. Hybridization or base pairing between nucleotides or nucleic acids, such as, for example, between the two strands of a double-stranded DNA molecule or between an oligonucleotide probe and a target are complementary.

The term “biological sample” refers to a sample obtained from an organism (e.g., a human patient) or from components (e.g., cells) of an organism. The sample may be of any biological tissue or fluid. The sample may be a “clinical sample” which is a sample derived from a patient. Such samples include, but are not limited to, sputum, blood, blood cells (e.g., white cells), amniotic fluid, plasma, semen, bone marrow, and tissue or fine needle biopsy samples, urine, peritoneal fluid, and pleural fluid, or cells therefrom. Biological samples may also include sections of tissues such as frozen sections taken for histological purposes. A biological sample may also be referred to as a “patient sample.”

A “protein” means a polymer of amino acid residues linked together by peptide bonds. The term, as used herein, refers to proteins, polypeptides, and peptides of any size, structure, or function. Typically, however, a protein will be at least six amino acids long. If the protein is a short peptide, it will be at least about 10 amino acid residues long. A protein may be naturally occurring, recombinant, or synthetic, or any combination of these. A protein may also comprise a fragment of a naturally occurring protein or peptide. A protein may be a single molecule or may be a multi-molecular complex. The term protein may also apply to amino acid polymers in which one or more amino acid residues is an artificial chemical analogue of a corresponding naturally occurring amino acid.

A “fragment of a protein,” as used herein, refers to a protein that is a portion of another protein. For example, fragments of proteins may comprise polypeptides obtained by digesting full-length protein isolated from cultured cells. In one embodiment, a protein fragment comprises at least about six amino acids. In another embodiment, the fragment comprises at least about ten amino acids. In yet another embodiment, the protein fragment comprises at least about sixteen amino acids.

As used herein, an “expression product” is a biomolecule, such as a protein, which is produced when a gene in an organism is expressed. An expression product may comprise post-translational modifications.

The term “metastasis” means the process by which cancer spreads from the place at which it first arose as a primary tumor to distant locations in the body. Metastasis also refers to cancers resulting from the spread of the primary tumor. For example, someone with colon cancer may show metastases in their liver or lungs.

The term “protein expression” refers to the process by which a nucleic acid sequence undergoes successful transcription and translation such that detectable levels of the amino acid sequence or protein are expressed.

The terms “protein expression profile” or “protein expression signature” refer to a group of proteins expressed by a particular cell or tissue type (e.g., neuron, coronary artery endothelium, or disease tissue), wherein presence of the proteins taken together or the differential expression of such proteins, is indicative/predictive of a certain condition.

The term “antibody” means an immunoglobulin, whether natural or partially or wholly synthetically produced. All derivatives thereof that maintain specific binding ability are also included in the term. The term also covers any protein having a binding domain that is homologous or largely homologous to an immunoglobulin binding domain. An antibody may be monoclonal or polyclonal. The antibody may be a member of any immunoglobulin class, including any of the human classes: IgG, IgM, IgA, IgD, and IgE.

The term “antibody fragment” refers to any derivative of an antibody that is less than full-length. In one aspect, the antibody fragment retains at least a significant portion of the full-length antibody's specific binding ability, specifically, as a binding partner. Examples of antibody fragments include, but are not limited to, Fab, Fab′, F(ab′)2, scFv, Fv, dsFv diabody, and Fd fragments. The antibody fragment may be produced by any means. For example, the antibody fragment may be enzymatically or chemically produced by fragmentation of an intact antibody or it may be recombinantly produced from a gene encoding the partial antibody sequence. Alternatively, the antibody fragment may be wholly or partially synthetically produced. The antibody fragment may comprise a single chain antibody fragment. In another embodiment, the fragment may comprise multiple chains that are linked together, for example, by disulfide linkages. The fragment may also comprise a multimolecular complex. A functional antibody fragment may typically comprise at least about 50 amino acids and more typically will comprise at least about 200 amino acids.

Determination of Gene Expression Profiles

The method used to identify and validate the present gene expression profiles indicative of whether a colon cancer patient's disease is likely to recur and/or metastasize is described below. Other methods for identifying gene and/or protein expression profiles are known; any of these alternative methods also could be used. See, e.g., Chen et al., NEJM, 356(1):11-20 (2007); Lu et al., PLOS Med., 3(12):e467 (2006); Wang et al., J. Clin. Oncol., 2299):1564 (2004); Golub et al., Science, 286:531-537 (1999).

The present method utilizes parallel testing in which, in one track, those genes are identified which are over-/under-expressed as compared to normal (non-cancerous) tissue and/or disease tissue from patients that experienced different outcomes; and, in a second track, those genes are identified comprising chromosomal insertions or deletions as compared to the same normal and disease samples. These two tracks of analysis produce two sets of data. The data are analyzed and correlated using an algorithm which identifies the genes of the gene expression profile (i.e., those genes that are differentially expressed in the cancer tissue of interest). Positive and negative controls may be employed to normalize the results, including eliminating those genes and proteins that also are differentially expressed in normal tissues from the same patients, and is disease tissue having a different outcome, and confirming that the gene expression profile is unique to the cancer of interest.

In the present instance, as an initial step, biological samples were acquired from patients afflicted with colorectal cancer. Tissue samples were obtained from patients diagnosed as having colon cancer, including samples of the primary resected tumor, metastatic lymph nodes and normal (undiseased) marginal colon tissue from each patient. Clinical information associated with each sample, including treatment with chemotherapeutic drugs, surgery, radiation or other treatment, outcome of the treatments and recurrence or metastasis of the disease, had been recorded in a database. Clinical information also includes information such as age, sex, medical history, treatment history, symptoms, family history, recurrence (yes/no), etc. Samples of normal (non-cancerous) tissue of different types (e.g., lung, brain, prostate) as well as samples of non-colon cancers (e.g., melanoma, breast cancer, ovarian cancer) were used as positive controls. Samples of normal undiseased colon tissue from a set of healthy individuals were used as positive controls, and colon tumor samples from patients whose cancer did recur/metastasize were used as negative controls.

Gene expression profiles (GEPs) then were generated from the biological samples based on total RNA according to well-established methods. Briefly, a typical method involves isolating total RNA from the biological sample, amplifying the RNA, synthesizing cDNA, labeling the cDNA with a detectable label, hybridizing the cDNA with a genomic array, such as the Affymetrix U133 GeneChip, and determining binding of the labeled cDNA with the genomic array by measuring the intensity of the signal from the detectable label bound to the array. See, e.g., the methods described in Lu, et al., Chen, et al. and Golub, et al., supra, and the references cited therein, which are incorporated herein by reference. The resulting expression data were input into a database.

MRNAs in the tissue samples can be analyzed using commercially available or customized probes or oligonucleotide arrays, such as cDNA or oligonucleotide arrays. The use of these arrays allows for the measurement of steady-state mRNA levels of thousands of genes simultaneously, thereby presenting a powerful tool for identifying effects such as the onset, arrest or modulation of uncontrolled cell proliferation. Hybridization and/or binding of the probes on the arrays to the nucleic acids of interest from the cells can be determined by detecting and/or measuring the location and intensity of the signal received from the labeled probe or used to detect a DNA/RNA sequence from the sample that hybridizes to a nucleic acid sequence at a known location on the microarray. The intensity of the signal is proportional to the quantity of cDNA or mRNA present in the sample tissue. Numerous arrays and techniques are available and useful. Methods for determining gene and/or protein expression in sample tissues are described, for example, in U.S. Pat. No. 6,271,002; U.S. Pat. No. 6,218,122; U.S. Pat. No. 6,218,114; and U.S. Pat. No. 6,004,755; and in Wang et al., J. Clin. Oncol., 22(9):1564-1671 (2004); Golub et al, (supra); and Schena et al., Science, 270:467-470 (1995); all of which are incorporated herein by reference.

The gene analysis aspect utilized in the present method investigates gene expression as well as insertion/deletion data. As a first step, RNA was isolated from the tissue samples and labeled. Parallel processes were run on the sample to develop two sets of data: (1) over-/under-expression of genes based on mRNA levels; and (2) chromosomal insertion/deletion data. These two sets of data were then correlated by means of an algorithm. Over-/under-expression of the genes in each cancer tissue sample were compared to gene expression in the normal (non-cancerous) samples and other control samples, and a subset of genes that were differentially expressed in the cancer tissue was identified. Preferably, levels of up- and down-regulation are distinguished based on fold changes of the intensity measurements of hybridized microarray probes. A difference of about 2.0 fold or greater is preferred for making such distinctions, or a p-value of less than about 0.05. That is, before a gene is said to be differentially expressed in diseased versus normal cells, the diseased cell is found to yield at least about 2 times greater or less intensity of expression than the normal cells. Generally, the greater the fold difference (or the lower the p-value), the more preferred is the gene for use as a diagnostic or prognostic tool. Genes selected for the gene signatures of the present invention have expression levels that result in the generation of a signal that is distinguishable from those of the normal or non-modulated genes by an amount that exceeds background using clinical laboratory instrumentation.

Statistical values can be used to confidently distinguish modulated from non-modulated genes and noise. Statistical tests can identify the genes most significantly differentially expressed between diverse groups of samples. The Student's t-test is an example of a robust statistical test that can be used to find significant differences between two groups. The lower the p-value, the more compelling the evidence that the gene is showing a difference between the different groups. Nevertheless, since microarrays allow measurement of more than one gene at a time, tens of thousands of statistical tests may be asked at one time. Because of this, it is unlikely to observe small p-values just by chance, and adjustments using a Sidak correction or similar step as well as a randomization/permutation experiment can be made. A p-value less than about 0.05 by the t-test is evidence that the expression level of the gene is significantly different. More compelling evidence is a p-value less then about 0.05 after the Sidak correction is factored in. For a large number of samples in each group, a p-value less than about 0.05 after the randomization/permutation test is the most compelling evidence of a significant difference.

Another parameter that can be used to select genes that generate a signal that is greater than that of the non-modulated gene or noise is the measurement of absolute signal difference. Preferably, the signal generated by the differentially expressed genes differs by at least about 20% from those of the normal or non-modulated gene (on an absolute basis). It is even more preferred that such genes produce expression patterns that are at least about 30% different than those of normal or non-modulated genes.

This differential expression analysis can be performed using commercially available arrays, for example, Affymetrix U133 GeneChip® arrays (Affymetrix, Inc.). These arrays have probe sets for the whole human genome immobilized on the chip, and can be used to determine up- and down-regulation of genes in test samples. Other substrates having affixed thereon human genomic DNA or probes capable of detecting expression products, such as those available from Affymetrix, Agilent Technologies, Inc. or Illumina, Inc. also may be used. Currently preferred gene microarrays for use in the present invention include Affymetrix U133 GeneChip® arrays and Agilent Technologies genomic cDNA microarrays. Instruments and reagents for performing gene expression analysis are commercially available. See, e.g., Affymetrix GeneChip® System. The expression data obtained from the analysis then is input into the database.

In the second arm of the present method, chromosomal insertion/deletion data for the genes of each sample as compared to samples of normal tissue was obtained. The insertion/deletion analysis was generated using an array-based comparative genomic hybridization (“CGH”). Array CGH measures copy-number variations at multiple loci simultaneously, providing an important tool for studying cancer and developmental disorders and for developing diagnostic and therapeutic targets. Microchips for performing array CGH are commercially available, e.g., from Agilent Technologies. The Agilent chip is a chromosomal array which shows the location of genes on the chromosomes and provides additional data for the gene signature. The insertion/deletion data from this testing is input into the database.

The analyses are carried out on the same samples from the same patients to generate parallel data. The same chips and sample preparation are used to reduce variability.

The expression of certain genes known as “reference genes” “control genes” or “housekeeping genes” also is determined, preferably at the same time, as a means of ensuring the veracity of the expression profile. Reference genes are genes that are consistently expressed in many tissue types, including cancerous and normal tissues, and thus are useful to normalize gene expression profiles. See, e.g., Silvia et al., BMC Cancer, 6:200 (2006); Lee et al., Genome Research, 12(2):292-297 (2002); Zhang et al., BMC Mol. Biol., 6:4 (2005). Determining the expression of reference genes in parallel with the genes in the unique gene expression profile provides further assurance that the techniques used for determination of the gene expression profile are working properly. The expression data relating to the reference genes also is input into the database. In a currently preferred embodiment, the following genes are used as reference genes: ACTB, GAPD, GUSB, RPLP0 and/or TRFC.

Data Correlation

The differential expression data and the insertion/deletion data in the database are correlated with the clinical outcomes information associated with each tissue sample also in the database by means of an algorithm to determine a gene expression profile for determining therapeutic efficacy of irinotecan, as well as late recurrence of disease and/or disease-related death associated with irinotecan therapy. Various algorithms are available which are useful for correlating the data and identifying the predictive gene signatures. For example, algorithms such as those identified in Xu et al., A Smooth Response Surface Algorithm For Constructing A Gene Regulatory Network, Physiol. Genomics 11:11-20 (2002), the entirety of which is incorporated herein by reference, may be used for the practice of the embodiments disclosed herein.

Another method for identifying gene expression profiles is through the use of optimization algorithms such as the mean variance algorithm widely used in establishing stock portfolios. One such method is described in detail in the patent application US Patent Application Publication No. 2003/0194734. Essentially, the method calls for the establishment of a set of inputs expression as measured by intensity) that will optimize the return (signal that is generated) one receives for using it while minimizing the variability of the return. The algorithm described in Irizarry et al., Nucleic Acids Res., 31:e15 (2003) also may be used. The currently preferred algorithm is the JMP Genomics algorithm available from JMP Software.

The process of selecting gene expression profiles also may include the application of heuristic rules. Such rules are formulated based on biology and an understanding of the technology used to produce clinical results, and are applied to output from the optimization method. For example, the mean variance method of gene signature identification can be applied to microarray data for a number of genes differentially expressed in subjects with cancer. Output from the method would be an optimized set of genes that could include some genes that are expressed in peripheral blood as well as in diseased tissue. If samples used in the testing method are obtained from peripheral blood and certain genes differentially expressed in instances of cancer could also be differentially expressed in peripheral blood, then a heuristic rule can be applied in which a portfolio is selected from the efficient frontier excluding those that are differentially expressed in peripheral blood. Of course, the rule can be applied prior to the formation of the efficient frontier by, for example, applying the rule during data pre-selection.

Other heuristic rules can be applied that are not necessarily related to the biology in question. For example, one can apply a rule that only a certain percentage of the portfolio can be represented by a particular gene or group of genes. Commercially available software such as the Wagner software readily accommodates these types of heuristics (Wagner Associates Mean-Variance Optimization Application). This can be useful, for example, when factors other than accuracy and precision have an impact on the desirability of including one or more genes.

As an example, the algorithm may be used for comparing gene expression profiles for various genes (or portfolios) to ascribe prognoses. The gene expression profiles of each of the genes comprising the portfolio are fixed in a medium such as a computer readable medium. This can take a number of forms. For example, a table can be established into which the range of signals (e.g., intensity measurements) indicative of disease is input. Actual patient data can then be compared to the values in the table to determine whether the patient samples are normal or diseased. In a more sophisticated embodiment, patterns of the expression signals (e.g., fluorescent intensity) are recorded digitally or graphically. The gene expression patterns from the gene portfolios used in conjunction with patient samples are then compared to the expression patterns. Pattern comparison software can then be used to determine whether the patient samples have a pattern indicative of recurrence of the disease. Of course, these comparisons can also be used to determine whether the patient is not likely to experience disease recurrence. The expression profiles of the samples are then compared to the profile of a control cell. If the sample expression patterns are consistent with the expression pattern for recurrence of cancer then (in the absence of countervailing medical considerations) the patient is treated as one would treat a relapse patient. If the sample expression patterns are consistent with the expression pattern from the normal/control cell then the patient is diagnosed negative for the cancer.

A method for analyzing the gene signatures of a patient to determine prognosis of cancer is through the use of a Cox hazard analysis program. The analysis may be conducted using S-Plus software (commercially available from Insightful Corporation). Using such methods, a gene expression profile is compared to that of a profile that confidently represents relapse (i.e., expression levels for the combination of genes in the profile is indicative of relapse). The Cox hazard model with the established threshold is used to compare the similarity of the two profiles (known relapse versus patient) and then determines whether the patient profile exceeds the threshold. If it does, then the patient is classified as one who will relapse and is accorded treatment such as adjuvant therapy. If the patient profile does not exceed the threshold then they are classified as a non-relapsing patient. Other analytical tools can also be used to answer the same question such as, linear discriminate analysis, logistic regression and neural network approaches. See, e.g., software available from JMP statistical software.

Numerous other well-known methods of pattern recognition are available. The following references provide some examples:
Weighted Voting: Golub, T R., Slonim, D K., Tamaya, P., Huard, C., Gaasenbeek, M., Mesirov, J P., Coller, H., Loh, L., Downing, J R., Caligiuri, M A., Bloomfield, C D., Lander, E S. Molecular classification of cancer: class discovery and class prediction by gene expression monitoring. Science 286:531-537, 1999.
Support Vector Machines: Su, A I., Welsh, J B., Sapinoso, L M., Kern, S G., Dimitrov, P., Lapp, H., Schultz, P G., Powell, S M., Moskaluk, C A., Frierson, H F. Jr., Hampton, G M. Molecular classification of human carcinomas by use of gene expression signatures. Cancer Research 61:7388-93, 2001. Ramaswamy, S., Tamayo, P., Rifkin, R., Mukherjee, S., Yeang, C H., Angelo, M., Ladd, C., Reich, M., Latulippe, E., Mesirov, J P., Poggio, T., Gerald, W., Loda, M., Lander, E S., Gould, T R. Multiclass cancer diagnosis using tumor gene expression signatures Proceedings of the National Academy of Sciences of the USA 98:15149-15154, 2001.
K-nearest Neighbors: Ramaswamy, S., Tamayo, P., Rifkin, R., Mukherjee, S., Yeang, C H., Angelo, M., Ladd, C., Reich, M., Latulippe, E., Mesirov, J P., Poggio, T., Gerald, W., Loda, M., Lander, E S., Gould, T R. Multiclass cancer diagnosis using tumor gene expression signatures Proceedings of the National Academy of Sciences of the USA 98:15149-15154, 2001.
Correlation Coefficients: van't Veer L J, Dai H, van de Vijver M J, He Y D, Hart A, Mao M, Peters H L, van der Kooy K, Marton M J, Witteveen A T, Schreiber G J, Kerkhoven R M, Roberts C, Linsley P S, Bernards R, Friend S H. Gene expression profiling predicts clinical outcome of breast cancer, Nature. 2002 Jan. 31; 415(6871):530-6.

The gene expression analysis identifies a gene expression profile (GEP) unique to the cancer samples, that is, those genes which are differentially expressed by the cancer cells. This GEP then is validated, for example, using real-time quantitative polymerase chain reaction (RT-qPCR), which may be carried out using commercially available instruments and reagents, such as those available from Applied Biosystems.

In the present instance, the results of the gene expression analysis showed that a number of genes were differentially expressed in colon cancer patients whose disease was unlikely to recur and/or metastasize. The genes having the highest level of differential expression included the following: AIK, MTOR, AKT, MAPK, MEK, 70S6, S6, HD60, IGFR/InR, IGFR1a, SSTR1, SSTR2, SSTR3, SSTR4 and SSTR5.

Determination of Protein Expression Profiles

Not all genes expressed by a cell are translated into proteins, therefore, once a GEP has been identified, it is desirable to ascertain whether proteins corresponding to some or all of the differentially expressed genes in the GEP also are differentially expressed by the same cells or tissue. Therefore, protein expression profiles (PEPs) are generated from the same cancer and control tissues used to identify the GEPs. PEPs also are used to validate the GEP in other colon cancer patients.

The preferred method for generating PEPs according to the present invention is by immunohistochemistry (IHC) analysis. In this method antibodies specific for the proteins in the PEP are used to interrogate tissue samples from cancer patients. Other methods for identifying PEPs are known, e.g. in situ hybridization (ISH) using protein-specific nucleic acid probes. See, e.g., Hofer et al., Clin. Can. Res., 11(16):5722 (2005); Volm et al., Clin. Exp. Metas., 19(5):385 (2002). Any of these alternative methods also could be used.

In the present instance, samples of colon tumor tissue, metastatic lymph nodes and normal margin colon tissue were obtained from patients afflicted with colon cancer who had undergone treatment of the primary tumor; these are the same samples used for identifying the GEP. The tissue samples as well as the positive and negative control samples were arrayed on tissue microarrays (TMAs) to enable simultaneous analysis. TMAs consist of substrates, such as glass slides, on which up to about 1000 separate tissue samples are assembled in array fashion to allow simultaneous histological analysis. The tissue samples may comprise tissue obtained from preserved biopsy samples, e.g., paraffin-embedded or frozen tissues. Techniques for making tissue microarrays are well-known in the art. See, e.g., Simon et al., BioTechniques, 36(1):98-105 (2004); Kallioniemi et al, WO 99/44062; Kononen et al., Nat. Med., 4:844-847 (1998). In the present instance, a hollow needle was used to remove tissue cores as small as 0.6 mm in diameter from regions of interest in paraffin embedded tissues. The “regions of interest” are those that have been identified by a pathologist as containing the desired diseased or normal tissue. These tissue cores then were inserted in a recipient paraffin block in a precisely spaced array pattern. Sections from this block were cut using a microtome, mounted on a microscope slide and then analyzed by standard histological analysis. Each microarray block can be cut into approximately 100 to approximately 500 sections, which can be subjected to independent tests.

For the present analysis, TMAs for the colon progression array were prepared using three tissue samples from each patient: one of colon tumor tissue, one from a lymph node and one of normal (undiseased) margin colon tissue (i.e., undiseased colon tissue surrounding the primary tumor site). The tumor tissues on the colon progression array included both recurrent and non-recurrent colon tumors, and lymph node tissues included both metastatic and normal (non-cancerous) lymph nodes. Control arrays also were prepared: a normal screening array containing normal tissue samples from healthy, cancer-free individuals was included as a negative control, and a cancer survey array including tumor tissues from cancer patients afflicted with cancers other than colon cancer, was used as a positive control.

Proteins in the tissue samples may be analyzed by interrogating the TMAs using protein-specific agents, such as antibodies or nucleic acid probes, such as oligonucleotides or aptamers. Antibodies are preferred for this purpose due to their specificity and availability. The antibodies may be monoclonal or polyclonal antibodies, antibody fragments, and/or various types of synthetic antibodies, including chimeric antibodies, or fragments thereof. Antibodies are commercially available from a number of sources (e.g., Abeam, Cell Signaling Technology or Santa Cruz Biotechnology), or may be generated using techniques well-known to those skilled in the art. The antibodies typically are equipped with detectable labels, such as enzymes, chromogens or quantum dots, which permit the antibodies to be detected. The antibodies may be conjugated or tagged directly with a detectable label, or indirectly with one member of a binding pair, of which the other member contains a detectable label. Detection systems for use with are described, for example, in the website of Ventana Medical Systems, Inc. Quantum dots are particularly useful as detectable labels. The use of quantum dots is described, for example, in the following references: Jaiswal et al., Nat. Biotechnol., 21:47-51 (2003); Chan et al., Curr. Opin. Biotechnol., 13:40-46 (2002); Chan et al., Science, 281:435-446 (1998).

The use of antibodies to identify proteins of interest in the cells of a tissue, referred to as immunohistochemistry (IHC), is well established. See, e.g., Simon et al., BioTechniques, 36(1):98 (2004); Haedicke et al., BioTechniques, 35(1):164 (2003), which are hereby incorporated by reference. The IHC assay can be automated using commercially available instruments, such as the Benchmark instruments available from Ventana Medical Systems, Inc.

In the present instance, the TMAs were contacted with antibodies specific for the proteins encoded by the genes identified in the gene expression study as being differentially expressed in colon cancer patients whose cancers had metastasized in order to determine expression of these proteins in each type of tissue. The antibodies used to interrogate the TMAs were selected based on the genes having the highest level of differential expression between recurrent and non-recurrent colon cancers.

The results of the IHC assay showed that in colon cancer patients whose cancers had not recurred/metastasized after treatment of the primary tumor, the following proteins were up-regulated: phospho-AIK, phospho-mTOR, phospho MAPK, phospho-MEK, phospho-S6, AKT, and SSTR1, compared with expression of these proteins in the colon tissue samples from those patients whose cancer had recurred and/or metastasized. Additionally, IHC analysis showed that a majority of these proteins were not up-regulated in the positive control tissue samples.

Assays

The present invention further comprises methods and assays for determining whether a colon cancer patient's disease is likely to recur/metastasize, or for predicting disease-related death associated with the cancer. According to one aspect, a formatted IHC assay can be used for determining if a colon cancer tumor exhibits the present GPEP. The assays may be formulated into kits that include all or some of the materials needed to conduct the analysis, including reagents (antibodies, detectable labels, etc.) and instructions.

The assay method of the invention comprises contacting a tumor sample from a colon cancer patient with a group of antibodies specific for some or all of the genes or proteins in the present GPEP, and determining the occurrence of up- or down-regulation of these genes or proteins in the sample. The use of TMAs allows numerous samples, including control samples, to be assayed simultaneously.

In a preferred embodiment, the method comprises contacting a tumor sample from a colon cancer patient and control samples with a group of antibodies specific for some or all of the proteins in the present GPEP, and determining the occurrence of up-regulation of these proteins. Up-regulation of some or all of the following proteins: phospho-AIK, phospho-mTOR, phospho MAPK, phospho-MEK, phospho-S6, AKT, and SSTR1, is indicative of the likelihood that the patient's disease will not recur/metastasize after treatment of the primary tumor. Preferably, at least about two, preferably between about four and six, and most preferably seven antibodies are used in the present method.

The method preferably also includes detecting and/or quantitating control or “reference proteins”. Detecting and/or quantitating the reference proteins in the samples normalizes the results and thus provides further assurance that the assay is working properly. In a currently preferred embodiment, antibodies specific for one or more of the following reference proteins are included: ACTB, GAPD, GUSB, RPLP0 and/or TRFC.

The present invention further comprises a kit containing reagents for conducting an IHC analysis of tissue samples or cells from colon cancer patients, including antibodies specific for at least about two of the proteins in the GPEP and for any reference proteins. The antibodies are preferably tagged with means for detecting the binding of the antibodies to the proteins of interest, e.g., detectable labels. Preferred detectable labels include fluorescent compounds or quantum dots, however other types of detectable labels may be used. Detectable labels for antibodies are commercially available, e.g. from Ventana Medical Systems, Inc.

Immunohistochemical methods for detecting and quantitating protein expression in tissue samples are well known. Any method that permits the determination of expression of several different proteins can be used. See. e.g., Signoretti et al., “Her-2-neu Expression and Progression Toward Androgen Independence in Human Prostate Cancer,” J. Natl. Cancer Instit., 92(23):1918-25 (2000); Gu et al., “Prostate stem cell antigen (PSCA) expression increases with high gleason score, advanced stage and bone metastasis in prostate cancer,” Oncogene, 19:1288-96 (2000). Such methods can be efficiently carried out using automated instruments designed for immunohistochemical (IHC) analysis. Instruments for rapidly performing such assays are commercially available, e.g., from Ventana Molecular Discovery Systems or Lab Vision Corporation. Methods according to the present invention using such instruments are carried out according to the manufacturer's instructions.

Protein-specific antibodies for use in such methods or assays are readily available or can be prepared using well-established techniques. Antibodies specific for the proteins in the GPEP disclosed herein can be obtained, for example, from Cell Signaling Technology, Inc, Santa Cruz Biotechnology, Inc. or Abeam.

The present invention is illustrated further by the following non-limiting Examples.

### EXAMPLES

A series of prognostic factors were tested in order to validate the efficacy of the gene/protein expression profile (GPEP) of the present invention for predicting the likelihood of recurrence of colon cancer following therapy. The expression levels of these factors, consisting of the seven (7) proteins in the present GPEP listed in Table 1, was determined by an immunohistochemical methodology in biopsy tissue samples obtained from colon cancer patients whose disease had recurred or metastasized, colon cancer patients whose disease had not recurred, and control samples.

Gene/Protein Expression Profile (GPEP):

Tissue samples were obtained from approximately ninety-two (92) patients diagnosed as having colon cancer, including samples of the primary resected tumor, lymph nodes and normal (undiseased) marginal colon tissue from each patient. The patients used in this study were suffering from various stages of colon cancer: adeno stages Dukes B1, B2, C and D. A total of 480 test tissue samples were used: forty cases from each stage, and three tissue samples (primary resected tumor, lymph nodes and normal marginal colon tissue) from each case. Approximately half of the patients had experienced recurrence or metastasis of their cancers within five-years after treatment of the primary tumor; the other half had not experienced recurrence or metastasis within five-years after treatment of the primary tumor.

In this study, formalin fixed paraffin embedded primary colon cancer specimens from colon cancer patients were evaluated for primary tumor size, metastasis, histologic grade and Duke's status. Using the techniques described above, a GEP was generated from these specimens comprising genes which were found to be differentially expressed in patents whose cancers had not recurred compared to patients whose cancer had recurred. He following genes comprised the GEP: AIK, MTOR, AKT, MAPK, MEK, 70S6, S6, HD60, IGFR/InR, IGFR1a, SSTR1, SSTR2, SSTR3, SSTR4 and SSTR5. Five reference genes were used to normalize the results: ACTB, GAPD, GUSB, RPLP0 and TRFC.

Tissue Microarrays:

Tissue microarrays were prepared using the colon adenocarinomas and normal (non-cancerous) colon tissue from patients described above having recurrent and non-recurrent colon cancers. TMAs also were prepared containing control samples; the control tissues are included to confirm that the GPEP is unique to non-recurrent colon cancer. A test array containing normal non-cancerous tissues was included as a control for antibody dilution, and also as another negative control. The TMAs used in this study are described in Table A:

TABLE A
Tissue Micro Arrays

| | |
|---|---|
| Colon Cancer | This array contained the patient samples obtained |
| Progression Array | from patients afflicted with recurrent/metastatic |
| | and non-recurrent colon adenocarcinoma. The |
| | samples include tumor tissue from the primary |
| | colon tumor, tissue from the surrounding lymph |
| | nodes and normal colon tissue samples from each |
| | patient. |
| Normal Screening | This array contained samples of normal (non- |
| Array | cancerous) tissue. The normal tissues in this array |
| | include lung, breast, ovarian, placenta, brain, |
| | pancreas, parotid gland, skin, colon, prostate and |
| | lymph node. This array was included as a |
| | negative control to confirm that the GPEP is |
| | unique to non-recurrent colon cancer tissue, i.e., |
| | that it does not occur in any normal tissues. |
| Cancer Screening | This array contained tumor samples for cancers |
| Survey Array | other than recurrent/metastatic colon cancer, |
| | including lung adeno, breast adeno, ovarian |
| | adeno, brain cancer (normal and glio), pancreas |
| | adeno, parotid gland cancer, melanoma, skin |
| | cancer, colon cancer (Dukes C and D) and |
| | prostate adeno. This array was included as a |
| | negative control to confirm that the GPEP is |
| | unique to non-recurrent colon cancer tissue, i.e., |
| | that it does not occur in any other cancer tissues. |
| Test Array | This array contained samples of the following |
| (TE-30 Array) | normal (non-cancerous) tissues: colon, liver, |
| | lung, prostate and breast. This array is included |
| | for antibody dilution and as a negative control to |
| | confirm that the GPEP is unique to non-recurrent |
| | colon cancer tissue, i.e., that it does not occur in |
| | any of these normal tissues. |

The TMAs were constructed according to the following procedure:

Tissue cores from donor block containing the patient tissue samples were inserted into a recipient paraffin block. These tissue cores are punched with a thin walled, sharpened borer. An X-Y precision guide allowed the orderly placement of these tissue samples in an array format.

Presentation: TMA sections were cut at 4 microns and are mounted on positively charged glass microslides. Individual elements were 0.6 mm in diameter, spaced 0.2 mm apart.

Elements: In addition to TMAs containing the recurrent and non-recurrent colon cancer samples, screening arrays were produced made up of cancer tissue samples other than recurrent colon cancer, 2 each from a different patient. Additional normal tissue samples were included for quality control purposes.

Specificity: The TMAs were designed for use with the specialty staining and immunohistochemical methods described below for gene expression screening purposes, by using monoclonal and polyclonal antibodies over a wide range of characterized tissue types.

Accompanying each array was an array locator map and spreadsheet containing patient diagnostic, histologic and demographic data for each element.

Immunohistochemical Staining

Immunohistochemical staining techniques were used for the visualization of tissue (cell) proteins present in the tissue samples. These techniques were based on the immunoreactivity of antibodies and the chemical properties of enzymes or enzyme complexes, which react with colorless substrate-chromogens to produce a colored end product. Initial immunoenzymatic stains utilized the direct method, which conjugated directly to an antibody with known antigenic specificity (primary antibody).

A modified labeled avidin-biotin technique was employed in which a biotinylated secondary antibody formed a complex with peroxidase-conjugated streptavidin molecules. Endogenous peroxidase activity was quenched by the addition of 3% hydrogen peroxide. The specimens then were incubated with the primary antibodies followed by sequential incubations with the biotinylated secondary link antibody (containing anti-rabbit or anti-mouse immunoglobulins) and peroxidase labeled streptavidin. The primary antibody, secondary antibody, and avidin enzyme complex is then visualized utilizing a substrate-chromogen that produces a brown pigment at the antigen site that is visible by light microscopy.

All of the TMAs were interrogated using a total of thirty-two antibodies specific for various tyrosine kinase pathway enzymes, including antibodies specific for both phosphorylated and non-phosphorylated forms of the protein. Antibodies were obtained from Cell Signaling Technology and Santa Cruz Biotechnology.

Automated Immunohistochemistry Staining Procedure (IHC):

1. Heat-induced epitope retrieval (HIER) using 10 mM Citrate buffer solution, pH 6.0, was performed as follows:

a. Deparaffinized and rehydrated sections were placed in a slide staining rack.

b. The rack was placed in a microwaveable pressure cooker; 750 ml of 10 mM Citrate buffer pH 6.0 was added to cover the slides.

c. The covered pressure cooker was placed in the microwave on high power for 15 minutes.

d. The pressure cooker was removed from the microwave and cooled until the pressure indicator dropped and the cover could be safely removed.

e. The slides were allowed to cool to room temperature, and immunohistochemical staining was carried out.

2. Slides were treated with 3% H2O2 for 10 min. at RT to quench endogenous peroxidase activity.

3. Slides were rinsed gently with phosphate buffered saline (PBS).

4. The primary antibodies were applied at the predetermined dilution (according to Cell Signaling Technology's Specifications) for 30 min at room temperature. Normal mouse or rabbit serum 1:750 dilution was applied to negative control slides.

5. Slides were rinsed with phosphate buffered saline (PBS).

6. Secondary biotinylated link antibodies* were applied for 30 min at room temperature.

7. Slides were rinsed with phosphate buffered saline (PBS).

8. The slides were treated with streptavidin-HRP (streptavidin conjugated to horseradish peroxidase)** for 30 min at room temperature.

9. Slides were rinsed with phosphate buffered saline (PBS).

10. The slides were treated with substrate/chromogen*** for 10 min at room temperature.

11. Slides were raised with distilled water.

12. Counter stain in Hematoxylin was applied for 1 min.

13. Slides were washed in running water for 2 min.

14. The slides were then dehydrated, cleared and the cover glass was mounted

*Secondary antibody: biotinylated anti-chicken and anti-mouse immunoglobulins in phosphate buffered saline (PBS), containing carrier protein and 15 mM sodium azide.

**Streptavidin-HRP in PBS containing carrier protein and anti-microbial agents from Ventana,

***Substrate-Chromogen is substrate-imidazole-HCl buffer pH 7.5 containing H202 and anti-microbial agents, DAB-3,3′-diaminobenzidine in chromogen solution from Ventana.

Experiment Notes:

All primary antibodies were titrated to dilutions according to manufacturer's specifications. Staining of TE30 Test Array slides (described in Table A) was performed with and without epitope retrieval (HIER). The slides were screened by a pathologist to determine the optimal working dilution. Pretreatment with HIER provided strong specific staining with little to no background. The above immunohistochemical staining was carried out using a Benchmark instrument from Ventana Medical Systems, Inc.

Scoring Criteria:

Staining was scored on a 0-3+ scale, with 0=no staining, and trace (tr) being less than 1+ but greater than 0. The scoring procedures are described in Signoretti et al., J. Nat. Cancer Inst., Vol. 92, No. 23, p. 1918 (December 2000) and Gu et al., Oncogene, 19, 1288-1296 (2000). Grades of 1+ to 3+ represent increased intensity of staining with 3+ being strong, dark brown staining. Scoring criteria was also based on total percentage of staining 0=0%, 1=less than 25%, 2=25-50% and 3=greater than 50%. The percent positivity and the intensity of staining for both nuclear and cytoplasmic as well as sub-cellular components were analyzed. Both the intensity and percentage positive scores were multiplied to produce one number 0-9. 3+ staining was determined from known expression of the antigen from the positive controls either breast adenocarcinoma and/or LNCAP cells.

Results

The data were preprocessed to average the antibody scores and remove any unknown or missing antibody scores. A univariate cox proportional hazard regression was preformed using SAS 8.2 software. The most statistically significant results are shown in Table B below.

TABLE B

| | | P Values for | |
|---|---|---|---|
| | Variable | Cox Regression | Hazard |
| Antibody Scores | Name | (univariate) | Ratio |
| Phospho-AIK (CST#3068) | AB1_cyto | 0.007 | 0.811 |
| Cyto Total Score | | | |
| Phospho-AIK (CST#3068) | AB1_nuclear | 0.43 | 0.945 |
| Nuclear Total Score | | | |
| Phospho-mTOR (CST#2971) | AB2_cyto | 0.003 | 0.797 |
| Cyto Total Score | | | |
| Phospho-mTOR (CST#2971) | AB2_nuclear | 0.5 | 0.958 |
| Nuclear Total Score | | | |
| Phospho-AKT (CST#9277) | AB3_cyto | 0.16 | 1.13 |
| Cyto Total Score | | | |
| Phospho-AKT (CST#9277) | AB3_nuclear | 0.93 | 1.005 |
| Nuclear Total Score | | | |
| Phospho AIK (CST#4718) | AB4_cyto | 0.93 | 0.992 |
| Cyto Total Score | | | |
| Phospho AIK (CST#4718) | AB4_nuclear | 0.17 | 1.07 |
| Nuclear Total Score | | | |
| Phospho MAPK (CST#9106) | AB5_cyto | 0.0042 | 0.841 |
| Cyto Total Score | | | |
| Phospho MAPK (CST#9106) | AB5_nuclear | .085 | 1.01 |
| Nuclear Total Score | | | |
| Phospho MEK (CST#9121) | AB6-cyto | 0.039 | 0.85 |
| Cyto Total Score | | | |
| Phospho MEK (CST#9121) | AB6_nuclear | 0.63 | 0.98 |
| Nuclear Total Score | | | |
| Phospho-p70S6 (CST#9206) | AB7_cyto | 0.93 | 1.008 |
| Cyto Total Score | | | |
| Phospho-p70S6 (CST#9206) | AB7_nuclear | 0.34 | 0.948 |
| Nuclear Total Score | | | |
| Phospho-S6 (CST#2211) | AB8_cyto | 0.07 | 0.857 |
| Cyto Total Score | | | |
| Phospho-S6 (CST#2211) | AB8_nuclear | 0.024 | 0.85 |
| Nuclear Total Score | | | |
| Total AKT (CST#9272) | AB9_cyto | 0.013 | 0.825 |
| Cyto Total Score | | | |
| Total AKT (CST#9272) | AB9_nuclear | 0.41 | 0.96 |
| Nuclear Total Score | | | |
| Total p70S6K (CST#9202) | AB10_cyto | 0.36 | 0.944 |
| Cyto Total Score | | | |
| Total p70S6K (CST#9202) | AB10_nuclear | 0.5 | 0.968 |
| Nuclear Total Score | | | |
| HD6 091801(#73362) | AB11_cyto | 0.36 | 1.057 |
| Cyto Total Score | | | |
| HD6 091801 (#73362) | AB11_nuclear | 0.65 | 0.936 |
| Nuclear Total Score | | | |
| p- IGFR1/lnR (CST#3021) | AB12_cyto | 0.57 | 0.953 |
| Cyto Total Score | | | |
| p- IGFR1/lnR (CST#3021) | AB12_nuclear | 0.08 | 0.872 |
| Nuclear Total Score | | | |
| Total IGFR1a CST#3022) | AB13_cyto | 0.68 | 1.034 |
| Cyto Total Score | | | |
| Total IGFR1a (CST#3022) | AB13_nuclear | 0.21 | 0.872 |
| Nuclear Total Score | | | |
| SSTR1 (SC#11604) | AB14_cyto | 0.031 | 0.8223 |
| Cyto Total Score | | | |
| SSTR2 (SC#11606) | AB15_cyto | 0.65 | 0.935 |
| Cyto Total Score | | | |
| SSTR3 (SC#11610) | AB16_cyto | 0.65 | 0.935 |
| Cyto Total Score | | | |
| SSTR4 (SC#11619) | AB17_cyto | 0.67 | 1.03 |
| Cyto Total Score | | | |
| SSTR5 (SC#11624) | AB18-cyto | 0.21 | 0.819 |
| Cyto Total Score | | | |

CST refers to Cell Signaling Technologies, and SC refers to Santa Cruz Biotechnology. The number in parenthesis is the catalog number of the antibody used in this experiment.

The antibodies having a p-value of 0.1 or less when tested vs. the dependent variable (here survival in months, which correlates with non-recurrence) are indicative of those proteins whose differential expression is most pronounced in non-recurrent colon cancer. These proteins, phospho-AIK, phospho-mTOR, phospho-MAPK, phospho-MEK, phosphoS6, AKT and SSTR1, comprise the present PEP. These seven proteins were not significantly over-expressed in those primary colon tumor samples derived from patients with recurrent and/or metastatic disease, or in metastatic lymph nodes. The over-expression of these seven proteins correlated strongly with those primary colon tumor samples from patients that did not experience a recurrence of their disease after five years. Of these seven proteins, phospho-MAPK and phospho-mTOR have the most significant prognostic value.

Positive, Negative and Isotype matched Controls and Reproducibility

Positive tissue controls were defined via western blot analysis using the antibodies listed in Table B. This experiment was performed to confirm the level of protein expression in each given control. Negative controls (Normal Screening Array and the Cancer Survey Array) also were defined by the same methodology.

Positive expression was confirmed using a Xenograft array. To make this array, SCID mice were injected with tumor cells derived from metastatic colon cancer cell lines SW480 and SW620 (both available from ATCC), and tumors were allowed to grow. The mice then were observed to determine the development of colon cancer. The tumors did not differentially express the proteins in the present GPEP.

Reproducibility:

All runs were grouped by antibody and tissue arrays which ensured that the runs were normalized, meaning that all of the tissue arrays were stained under the same conditions with the same antibody on the same run. A test array containing thirty negative control samples (TE 30) comprising non-cancerous tissues derived from several organs also was provided. The staining of this TE 30 array was compared to the previous antibody run and scored accordingly. The reproducibility was compared and validated.

Results:

In tumor samples obtained from those patients whose colon cancer had not recurred or metastasized after five years, the following proteins were up-regulated: phospho-AIK, phospho-mTOR, phospho-MAPK, phospho-MEK, phosphoS6, AKT and SSTR1, compared with expression of these proteins in colon cancers that had recurred and in metastatic lymph nodes. In contrast, most of these proteins were not up-regulated in the positive or negative control tissue samples.

These results show that the present protein expression profile is indicative of the likelihood that a patient's colon cancer will recur or metastasize. These data also support a potential role for this signature as a determinant of the activity of these TK enzymes in colon tumor cells, and expression as novel biomarkers for predicting the likelihood of recurrence and/or metastasis in colon cancer patients.

1. A method of determining if a colon cancer patient's colon cancer is likely to recur, comprising
a. obtaining a tumor sample from the colon cancer patient; and
b. determining the expression levels in the sample of at least about two proteins selected from the group consisting of: phospho-AIK, phospho-mTOR, phospho MAPK, phospho-MEK, phospho-S6; AKT, and SSTR1.

2. The method of claim 1 wherein step (b) comprises determining the expression level of at least phospho-MAPK and phospho-mTOR.

3. The method of claim 1 wherein the expression of the proteins is up-regulated in patients whose colon cancer is not likely to recur compared to expression of these proteins in patients whose cancer is likely to recur.

4. The method of claim 1 further comprising means for determining the expression level of at least one reference protein.

5. The method of claim 4 wherein the reference protein is selected from the group consisting of: ACTB, GAPD, GUSB, RPLP0 and TRFC.

6. The method of claim 1 wherein step (b) is carried out using immunohistochemistry.

7. An assay for determining if a colon cancer patient's colon cancer is likely to recur, comprising means for determining the expression levels in a tumor cell or tumor tissue of said colon cancer patient of at least two proteins selected from the group consisting of: phospho-mTOR, phospho-pTEN, phospho-MAPK, phospho-IGFR/InR and phospho-EGFR.

8. The assay of claim 7 wherein the expression of the proteins is up-regulated in patients whose colon cancer is not likely to recur compared to expression of these proteins in patients whose cancer is likely to recur.

9. The assay of claim 7 wherein the at least two proteins comprise phospho-MAPK and phospho-mTOR.

10. The assay of claim 7 further comprising means for determining the expression level of at least one reference protein.

11. The assay of claim 10 wherein the reference protein is selected from the group consisting of: ACTB, GAPD, GUSB, RPLP0 and TRFC.

12. A method of determining if a colon cancer patient's colon cancer is likely to recur, comprising:
a. obtaining a tumor sample from the colon cancer patient; and
b. determining the expression levels in the sample of at least about two genes selected from the group consisting of: AIK, mTOR, MAPK, MEK, S6; AKT, and SSTR1.

13. The method of claim 12 wherein step (b) comprises determining the expression of at least MAPK and mTOR.

14. The method of claim 12 wherein the expression of the genes is up-regulated in patients whose colon cancer is not likely to recur compared to expression of these genes in patients whose cancer is likely to recur.

15. The method of claim 12 further comprising means for determining the expression level of at least one reference gene.

16. The method of claim 15 wherein the reference gene is selected from the group consisting of: ACTB, GAPD, GUSB, RPLP0 and TRFC.

17-20. (canceled)
